(19) United States
(12) Patent Application Publication
(10) Pub. No.: US 2022/0088144 A1
(43) Pub. Date: Mar. 24, 2022

(54) SGLT-2 INHIBITORS OR IL-1R ANTAGONISTS FOR REDUCTION OF HYPOGLYCAEMIA AFTER BARIATRIC SURGERY

(71) Applicant: UNIVERSITÄTSSPITAL BASEL, Basel (CH)

(72) Inventor: Marc DONATH, Basel (CH)

(73) Assignee: UNIVERSITÄTSSPITAL BASEL, Basel (CH)

(21) Appl. No.: 17/422,193

(22) PCT Filed: Jan. 13, 2020
(86) PCT No.: PCT/EP2020/050700

§ 371 Date: Jul. 11, 2021

(30) Foreign Application Priority Data
Jan. 11, 2019 (EP) 19151525.3
Jun. 25, 2019 (EP) 19182437.4

(51) Int. Cl. A61K 38/20; A61K 31/7048; A61K 31/7042; A61K 31/351; A61K 31/382; C07K 16/24; C07K 16/28; A61P 3/08

(52) U.S. Cl. CPC A61K 38/2006; A61K 31/7048; A61K 31/7042; A61P 3/08; A61K 31/382; C07K 16/245; C07K 16/2866; A61K 31/351

(57) ABSTRACT

The present invention relates to the use of SGLT-2 inhibitors and/or an interleukin-1-receptor antagonist or a non-agonist antibody reactive to IL-1 or IL-1R in the treatment of prevention of symptomatic hypoglycaemia after bariatric surgery.

The present invention relates to the use of SGLT-2 inhibitors, particularly empagliflozin, and/or interleukin-1-receptor antagonists or non-agonist antibodies reactive to interleukin-1ß or IL-1 receptor in the treatment of prevention of hypoglycaemia, particularly hypoglycaemia after bariatric surgery.

### BACKGROUND OF THE INVENTION

Obesity and its associated comorbidities are a major global burden with increasing prevalence that rose from 29% to 37% within the last three decades and is estimated to reach a prevalence of 40-50% of the adult population in the United States by 2030. Obesity and its associated comorbidities lead to a 5 to 20-years lower life expectancy compared to their age- and gender-matched non-obese population. For patients with morbid obesity or obesity and metabolic comorbidities bariatric surgery is the most effective treatment option. Bariatric surgery comprises at present mainly gastric sleeve (GS) and Roux-Y-gastric bypass (RYGB) aside biliopancreatic diversion (BPD) and gastric banding. Bariatric surgery is associated with a low short-term mortality and may also be associated with a reduction in all-cause, cardiovascular as well as cancer-related mortality. Bariatric surgery has several positive effects on metabolic changes in obese subjects such as improvement of diabetes, arterial hypertension, hyperlipidaemia, lowered incidence of microvascular disease, and prevention of gout and hyperuricemia as well as a reduction of atrial fibrillation and reduced risk of female-specific cancer, especially in women with hyperinsulinemia at baseline.

However, several surgical and medical conditions may occur after bariatric surgery and impair quality of life. Even though most patients report an improved well-being after RYGB, the prevalence of symptoms after bariatric surgery were reported by 88.6% of the patients with more than two third of them contacting health care institutions. Most commonly abdominal pain (34.2%), fatigue (34.1%) and anemia (27.7%) were reported. Predictors of symptoms were patients younger than 35 years of age, female sex, smoking and pre-existing symptoms before operation.

The post-prandial reported Fatigue may be in part or fully caused by hypoglycemic episodes given the fact that it often consists of unspecific symptoms and thereby is frequently under diagnosed. The prevalence of post-prandial hypoglycaemia in bariatric patients—also referred as late dumping—ranges from 0.5% severe episodes up to 34.2% and 56% and is increasingly recognized as a post-bariatric complication. Thus, lowered life-quality and even fatal outcomes were reported. This hypoglycemic condition seems to be triggered by a rapid increase of plasma glucose followed by an increased glucose-lowering reaction that is governed by insulin, insulinotropic incretins, polypeptides, changes in gut microbiota and bile acid composition, or other abnormal counter regulatory mechanisms. Hypoglycaemia itself may lead to increased hunger, carbohydrate ingestion and following weight regain. Risk factors for spontaneously self-reported postprandial hypoglycaemia after bariatric surgery are lower pre-surgery plasma glucose concentrations, higher insulin sensitivity, better β-cell glucose sensitivity as well as longer post-bariatric period, lack of diabetes and female sex. However, the exact mechanisms leading to hypoglycaemia still need to be elucidated and therapeutic options are warranted. So far, the mainstay in the treatment of post-prandial hypoglycaemia consists of dietary intervention with carbohydrate control with 5-6 meals and reduced glycaemic index. Medical therapeutic opportunities comprise acarbose (α-glucosidase inhibitor), calcium channel blockers (diazoxide), somatostatin analogues (e.g. ocreotide, pasireotide) or exogenous GLP-1 but warrant further examination.

Based on the above-mentioned state of the art, the objective of the present invention is to provide means and methods to treat hypoglycaemia in patients after bariatric surgery. This objective is attained by the subject-matter of the independent claims of the present specification.

### SUMMARY OF THE INVENTION

A first aspect of the invention relates to the use of an SGLT-2 inhibitor for treatment of prevention of hypoglycaemia after bariatric surgery.

A second aspect of the invention relates to the use of an interleukin-1-receptor antagonist for treatment of prevention of hypoglycaemia after bariatric surgery.

A third aspect of the invention relates to the use of a non-agonist antibody or antibody-like molecule specifically binding to one of
IL-1β or
IL-1 receptor type I

in treatment of prevention of hypoglycaemia after bariatric surgery.

A fourth aspect of the invention relates to the use of an NLRP3 inhibitor in treatment of prevention of hypoglycaemia after bariatric surgery.

### BRIEF DESCRIPTION OF THE FIGURES

FIG. 1 Schedule at study dates.

FIG. 2 Study Design.

FIG. 3 Comparison of insulin secretion index between control and interventions.

FIG. 4 Comparison of whole-body insulin sensitivity index between control and interventions.

FIG. 5 Metabolic parameters in patients after bariatric surgery undergoing a mixed meal test upon treatment with empagliflozin or anakinra.

FIG. 6 Monocytes taken from patients on days with hypoglycemia show upregulation of inflammatory pathways.

FIG. 7 Food-intake induces gene expression of pattern-recognition receptors, as well as immune and metabolic pathways in patients post-bariatric surgery.

FIG. 8 Gene expression in response to hypoglycemia appears to be an exaggeration of the response to food-intake.

FIG. 9 Ex vivo cytokine secretion in meal- and hypoglycemia-preconditioned monocytes.

FIG. 10 Hypoglycemic events and plasma glucose and for patient 3.

FIG. 11 Prevention of hypoglycemia by empagliflozin or anakinra is independent of changes in GLP-1 or glucagon.

FIG. 12 Representative Plots detailing the sorting strategy employed for the isolation of bulk monocytes.

FIG. 13 GSEA enrichment analysis of an “Endotoxin Tolerant Gene Signature” in hypoglycemic patients.

FIG. 14 Representative continuous glucose monitoring report of a 31-year old female patient before and during treatment with 10 mg empagliflozin daily.

FIG. 15 Edinburgh Hypoglycemia Scale, Sigstad Score and Standford Sleepiness Scale Score by treatment.

### DETAILED DESCRIPTION OF THE INVENTION

### Terms and Definitions

Unless defined otherwise, all technical and scientific terms used herein have the same meaning as commonly understood by one of ordinary skill in the art (e.g., in cell culture, molecular genetics, nucleic acid chemistry, hybridization techniques and biochemistry). Standard techniques are used for molecular, genetic and biochemical methods (see generally, Sambrook et al., Molecular Cloning: A Laboratory Manual, 4th ed. (2012) Cold Spring Harbor Laboratory Press, Cold Spring Harbor, N.Y. and Ausubel et al., Short Protocols in Molecular Biology (2002) 5th Ed, John Wiley & Sons, Inc.) and chemical methods.

The term bariatric surgery in the context of the present specification relates to the most common bariatric surgery procedures which are gastric bypass, sleeve gastrectomy, adjustable gastric band, and biliopancreatic diversion with duodenal switch.

The term SGLT-2 inhibitors in the context of the present specification relates to all drugs of this class including but not limited to empagliflozin (CAS No. 864070-44-0), canagliflozin (CAS No. 842133-18-0), dapagliflozin (CAS No. 461432-26-8), ertugliflozin (CAS No. 1210344-57-2), ipragliflozin (CAS No. 761423-87-4), luseogliflozin (CAS No. 898537-18-3), sotagliflozin (CAS No. 1018899-04-1), and tofogliflozin (CAS No. 1201913-82-7).

The term interleukin-1-receptor antagonists or non-agonist antibodies reactive to interleukin-1ß or to IL1 receptor type I in the context of the present specification relates to drugs blocking the production or action of IL-1, IL-1β and IL-1α. This includes the IL-1 receptor antagonist IL-1Ra (anakinra), antibodies blocking the action of IL-1β and IL-1α, and small molecules blocking the production of IL-1β, such as NLRP3 inhibitors.

In the context of the present specification, the term antibody refers to whole antibodies including but not limited to immunoglobulin type G (IgG), type A (IgA), type D (IgD), type E (IgE) or type M (IgM), any antigen binding fragment or single chains thereof and related or derived constructs. A whole antibody is a glycoprotein comprising at least two heavy (H) chains and two light (L) chains inter-connected by disulfide bonds. Each heavy chain is comprised of a heavy chain variable region (V_{H}) and a heavy chain constant region (C_{H}). The heavy chain constant region is comprised of three domains, C_{H}1, C_{H}2 and C_{H}3. Each light chain is comprised of a light chain variable region (abbreviated herein as V_{L}) and a light chain constant region (C_{L}). The light chain constant region is comprised of one domain, C_{L}. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component of the classical complement system. Similarly, the term encompasses a so-called nanobody or single domain antibody, an antibody fragment consisting of a single monomeric variable antibody domain.

The term antibody-like molecule in the context of the present specification refers to a molecule capable of specific binding to another molecule or target with high affinity, particularly characterized by a Kd≤10E-8 mol/l. An antibody-like molecule binds to its target similarly to the specific binding of an antibody. The term antibody-like molecule encompasses a repeat protein, such as a designed ankyrin repeat protein (Molecular Partners, Zürich), an engineered antibody mimetic proteins exhibiting highly specific and high-affinity target protein binding (see US2012142611, US2016250341, US2016075767 and US2015368302, all of which are incorporated herein by reference). The term antibody-like molecule further encompasses, but is not limited to, a polypeptide derived from armadillo repeat proteins, a polypeptide derived from leucine-rich repeat proteins and a polypeptide derived from tetratricopeptide repeat proteins.

The term antibody-like molecule further encompasses a specifically binding polypeptide derived from a protein A domain, fibronectin domain FN3, consensus fibronectin domains, a lipocalins (see Skerra, Biochim. Biophys. Acta 2000, 1482(1-2):337-50), a polypeptide derived from a Zinc finger protein (see Kwan et al. Structure 2003, 11(7):803-813), Src homology domain 2 (SH2) or Src homology domain 3 (SH3), a PDZ domain, gamma-crystallin, ubiquitin, a cysteine knot polypeptide or a knottin, cystatin, Sac7d, a triple helix coiled coil (also known as alphabody), a Kunitz domain or a Kunitz-type protease inhibitor and a carbohydrate binding module 32-2.

As used herein, the term treating or treatment of any disease or disorder (e.g. cancer) refers in one embodiment, to ameliorating the disease or disorder (e.g. slowing or arresting or reducing the development of the disease or at least one of the clinical symptoms thereof). In another embodiment “treating” or “treatment” refers to alleviating or ameliorating at least one physical parameter including those which may not be discernible by the patient. In yet another embodiment, “treating” or “treatment” refers to modulating the disease or disorder, either physically, (e.g., stabilization of a discernible symptom), physiologically, (e.g., stabilization of a physical parameter), or both. Methods for assessing treatment and/or prevention of disease are generally known in the art, unless specifically described herein below.

The term having substantially the same biological activity in the context of the present invention relates to the main functions of the interleukin-1 receptor antagonist protein, i.e. anti-hypoglycaemic activity.

### DETAILED DESCRIPTION OF THE INVENTION

Obesity is associated with a chronic activation of the IL-1β pathway which is increased following ingestion of a meal. Furthermore, IL-1β acutely increases insulin secretion. Therefore, it was investigated whether inhibition of elevated endogenous IL-1β levels with the IL-1 receptor antagonist anakinra (Kineret®) reduces circulating insulin levels in patients with hypoglycaemia after bariatric surgery.

To the inventors' knowledge, no clinical data have been published investigating the effect of SGLT2-inhibitors nor anakinra or any other drug impacting on the IL-1R/IL-1ß interaction on hypoglycaemia in patients after bariatric surgery.

A first aspect of the invention relates to the use of an SGLT-2 inhibitor in treatment of prevention of hypoglycaemia after bariatric surgery.

In certain embodiments, the SGLT-2 inhibitor is selected from empagliflozin, canagliflozin, dapagliflozin, ertugliflozin, ipragliflozin, luseogliflozin, sotagliflozin, and tofogliflozin. In certain embodiments, the SGLT-2 inhibitor is empagliflozin.

Empagliflozin (CAS No. 864070-44-0; marketed as Jardiance®; Boehringer Ingelheim) is a highly selective, reversible inhibitor of the sodium glucose co-transporter 2 (SGLT2) and a novel treatment modality as monotherapy or in combination with metformin, sulfonylurea or insulin in patients with type 2 diabetes mellitus. SGLT2 is predominantly expressed in the proximal tubule of the kidney and reabsorbs approximately 90 percent of the filtered glucose leading to an increased glucosuria (64 g glucose per day). In Switzerland, 10 mg tablets are available, containing the active substance empagliflozinum and the adjuvant lactosum monohydricum. The effect of glucose-excretion relies on glucose plasma concentration as well as glomerular filtration rate and rises after the intake of the first dose of empagliflozin (Jardiance®) with a median Tₘₐₓ of 1.5 h and a mean Plasma-AUC in the steady state of 1.87 nmol·h/l allowing once daily dosing.

Contraindications for its use are a history of serious hypersensitivity to empagliflozin or any component of the formulation. Clinical trials have shown an acceptable safety profile for empagliflozin. Recently, it was shown for patients with type 2 diabetes and a high cardiovascular risk who received empagliflozin as compared with placebo, had a significant lower rate of the primary composite cardiovascular outcome and of death from any cause when the study drug was added to standard care.

In mid- and long-term use, adverse events such as pollakisuria, increased thirst and the occurrence of urinary- or genital tract infections were reported. Since glucose-excretion is dependent on the filtered load, it has a low propensity to cause hypoglycemia. Mild hypoglycemias have been reported in the combined treatment with insulin or sulfonylurea.

In certain embodiments, the SGLT-2 inhibitor is administered once a day. In certain embodiments, the SGLT-2 inhibitor is administered three to one hours before a meal. In certain embodiments, the SGLT-2 inhibitor is administered two hours before a meal.

In certain embodiments, the SGLT-2 inhibitor is administered at a dose of 5-20 mg/day. In certain embodiments, the SGLT-2 inhibitor is administered at a dose of 10 mg/day.

Empagliflozin is rapidly absorbed after oral administration and maximum plasma concentration is observed after 1.5 h of administration, leading to glucosuria for at least 24 hours. Empagliflozin 10 mg is approved for treatment of diabetes mellitus type 2 in Switzerland, and is well tolerated with a favourable safety profile. Therefore, this standard dose was used.

A second aspect of the invention relates to an interleukin-1-receptor antagonist for use in treatment of prevention of hypoglycaemia after bariatric surgery.

In certain embodiments, the interleukin-1-receptor antagonist is anakinra.

Anakinra (CAS NO 143090-92-0; Kineret®; r-metHuIL-1ra, Swedish Orphan Biovitrum AB) is a recombinant, non-glycosylated form of the human interleukin-1 receptor antagonist (IL-1 Ra). It is commonly administered as a 100 mg/0.67 ml solution for SC injection. Anakinra differs from native human IL-1Ra in that it has the addition of a single methionine residue at its amino terminus. Anakinra consists of 153 amino acids and has a molecular weight of 17.3 kilodaltons, and Anakinra is produced by recombinant DNA technology using an E. coli bacterial expression system. Other forms of this drug that may vary slightly in structure or administration form while having the same biological effect may be used instead.

Mild and transient injection reactions might occur in 20-50% after 2-4 weeks of injections. In the present study, only a single injection was applied and therefore injection site reactions were not expected.

Anakinra is approved for the treatment of rheumatoid arthritis in the US by the FDA as well as in Europe by the EMA and has an acceptable risk/benefit profile in this indication, with more than 100,000 patients treated.

In certain embodiments, the interleukin-1-receptor antagonist is administered once a day. In certain embodiments, the interleukin-1-receptor antagonist is administered four to two hours before a meal. In certain embodiments, the interleukin-1-receptor antagonist is administered three hours before a meal.

In certain embodiments, the interleukin-1-receptor antagonist is administered at a dose of 50-200 mg/day. In certain embodiments, the interleukin-1-receptor antagonist is administered at a dose of 100 mg/day.

Anakinra is rapidly absorbed after subcutaneous administration and maximum plasma concentration is observed after 4 h of administration. Anakinra 100 mg is approved for the treatment of rheumatoid arthritis. Therefore, this standard dose was used.

To minimize potential bias in the evaluation of the possible effect of empagliflozin as well as anakinra on hypoglycaemia in patients after bariatric surgery, a double blind placebo-controlled design was chosen.

A third aspect of the invention relates to a non-agonist antibody or antibody-like molecule specifically binding to one of
IL-1β or
IL-1 receptor type I

for use in treatment of prevention of hypoglycaemia after bariatric surgery.

A fourth aspect of the invention relates to a NLRP3 inhibitor for use in treatment of prevention of hypoglycaemia after bariatric surgery. The NLRP3 inhibitor may be selected from, but is not limited to, MCC950 (CAS No. 210826-40-7), MNS (3,4-methylenedioxy-beta-nitrostyrene, CAS No. 1485-00-3), CY-09 (CAS No. 1073612-91-5), tranilast (CAS No. 53902-12-8), OLT1177 (CAS No. 54863-37-5), oridonin (CAS No. 28957-04-2), IFM-2427 (Novartis), Bay 11-7082 (CAS No. 19542-67-7) and β-hydroxybutyrate.

In certain embodiments, said NLRP3 inhibitor is administered once a day. In certain embodiments, said NLRP3 inhibitor is administered three to one hours before a meal. In certain embodiments, said NLRP3 inhibitor is administered two hours before a meal.

Similarly within the scope of the present invention is a method or treating hypoglycaemia after bariatric surgery in a patient in need thereof, comprising administering to the patient an SGLT-2 inhibitor or an interleukin-1 receptor antagonist protein according to the above description.

Similarly, a dosage form for the prevention or treatment of hypoglycaemia after bariatric surgery is provided, comprising a SGLT-2 inhibitor or an interleukin-1 receptor antagonist protein according to any of the above aspects or embodiments of the invention.

The skilled person is aware that any specifically mentioned drug may be present as a pharmaceutically acceptable salt of said drug. Pharmaceutically acceptable salts comprise the ionized drug and an oppositely charged counterion. Non-limiting examples of pharmaceutically acceptable anionic salt forms include acetate, benzoate, besylate, bitatrate, bromide, carbonate, chloride, citrate, edetate, edisylate, embonate, estolate, fumarate, gluceptate, gluconate, hydrobromide, hydrochloride, iodide, lactate, lactobionate, malate, maleate, mandelate, mesylate, methyl bromide, methyl sulfate, mucate, napsylate, nitrate, pamoate, phosphate, diphosphate, salicylate, disalicylate, stearate, succinate, sulfate, tartrate, tosylate, triethiodide and valerate. Non-limiting examples of pharmaceutically acceptable cationic salt forms include aluminium, benzathine, calcium, ethylene diamine, lysine, magnesium, meglumine, potassium, procaine, sodium, tromethamine and zinc.

In certain embodiments, the compound of the invention according to any of the aspects and embodiments disclosed herein is used after bariatric surgery selected from Roux-Y-gastric bypass, vertical banded gastroplasty surgery, adjustable gastric band, and partial ileal bypass surgery. In certain embodiments, the bariatric surgery is Roux-Y-gastric bypass.

The term hypoglycaemia includes, but is not limited to, symptomatic hypoglycaemia.

Dosage forms may be for enteral administration, such as nasal, buccal, rectal, transdermal or oral administration, or as an inhalation form or suppository. Alternatively, parenteral administration may be used, such as subcutaneous, intravenous, intrahepatic or intramuscular injection forms. Optionally, a pharmaceutically acceptable carrier and/or excipient may be present.

The invention further encompasses the following items:
1. An SGLT-2 inhibitor for use in treatment or prevention of postprandial hypoglycaemia.
2. The SGLT-2 inhibitor for use in treatment or prevention of postprandial hypoglycaemia according to item 1, wherein said SGLT-2 inhibitor is selected from empagliflozin, canagliflozin, dapagliflozin, ertugliflozin, ipragliflozin, luseogliflozin, sotagliflozin, and tofogliflozin, particularly wherein said SGLT-2 inhibitor is empagliflozin.
3. The SGLT-2 inhibitor for use in treatment or prevention of postprandial hypoglycaemia according to any one of items 1 or 2, wherein said SGLT-2 inhibitor is administered once a day, particularly three to one hours before a meal, more particularly two hours before a meal.
4. The SGLT-2 inhibitor for use in treatment or prevention of postprandial hypoglycaemia according to any one of items 1 to 3, wherein said SGLT-2 inhibitor is administered at a dose of 5-20 mg/day.
5. The SGLT-2 inhibitor for use in treatment or prevention of postprandial hypoglycaemia according to any one of items 1 to 4, wherein the hypoglycaemia is symptomatic hypoglycaemia.
6. An interleukin-1-receptor antagonist for use in treatment or prevention of postprandial hypoglycaemia.
7. The interleukin-1-receptor antagonist for use in treatment or prevention of postprandial hypoglycaemia according to item 6, wherein said interleukin-1-receptor antagonist is anakinra.
8. The interleukin-1-receptor antagonist for use in treatment or prevention of postprandial hypoglycaemia according to any one of items 6 to 7, wherein said interleukin-1-receptor antagonist is administered once a day, particularly four to two hours before a meal, more particularly three hours before a meal.
9. The interleukin-1-receptor antagonist for use in treatment or prevention of postprandial hypoglycaemia according to any one of items 6 to 8, wherein said interleukin-1-receptor antagonist is administered at a dose of 50-200 mg/day.
10. The interleukin-1-receptor antagonist for use in treatment or prevention of postprandial hypoglycaemia according to any one of items 6 to 11, wherein the hypoglycaemia is symptomatic hypoglycaemia.
11. A non-agonist antibody or antibody-like molecule specifically binding to one of
   IL-1β or
   IL-1 receptor type I
   for use in treatment or prevention of postprandial hypoglycaemia, particularly symptomatic hypoglycaemia.
12. A NLRP3 inhibitor for use in treatment or prevention of postprandial hypoglycaemia, particularly symptomatic hypoglycaemia.
13. The NLRP3 inhibitor for use in treatment or prevention of postprandial hypoglycaemia according to item 12, wherein said NLRP3 inhibitor is selected from MCC950, MNS, CY-09, tranilast, OLT1177, oridonin, IFM-2427, Bay 11-7082 and β-hydroxybutyrate.
14. The NLRP3 inhibitor for use in treatment or prevention of postprandial hypoglycaemia according to any one of items 12 to 13, wherein said NLRP3 inhibitor is administered once a day, particularly three to one hours before a meal, more particularly two hours before a meal.
15. A NLRP3 inhibitor for use in treatment or prevention of hypoglycaemia, particularly symptomatic hypoglycaemia, after bariatric surgery.
16. The NLRP3 inhibitor for use in treatment or prevention of after bariatric surgery according to item 15, wherein said bariatric surgery is selected from Roux-Y-gastric bypass, vertical banded gastroplasty surgery, adjustable gastric band, and partial ileal bypass surgery, particularly wherein said bariatric surgery is Roux-Y-gastric bypass.
17. The NLRP3 inhibitor for use in treatment or prevention of hypoglycaemia after bariatric surgery according to item 15 or 16, wherein said NLRP3 inhibitor is selected from MCC950, MNS, CY-09, tranilast, OLT1177, oridonin, IFM-2427, Bay 11-7082 and β-hydroxybutyrate.
18. The NLRP3 inhibitor for use in treatment or prevention of hypoglycaemia after bariatric surgery according to any one of items 15 to 17, wherein said NLRP3 inhibitor is administered once a day, particularly three to one hours before a meal, more particularly two hours before a meal.

The data contained herein below demonstrate that the agents identified in the above items are effective for postprandial hypoglycemia, regardless of whether they have undergone bariatric surgery or not. The selection of patients having undergone bariatric surgery simply represents a collective of particular clinical need, for whom conducting a study was ethically justified. The conclusions being drawn from the study's results, however, can be extended to any postprandial situation.

Wherever alternatives for single separable features are laid out herein as “embodiments”, it is to be understood that such alternatives may be combined freely to form discrete embodiments of the invention disclosed herein.

The invention is further illustrated by the following examples and figures, from which further embodiments and advantages can be drawn. These examples are meant to illustrate the invention but not to limit its scope.

### DESCRIPTION OF THE FIGURES

FIG. 1 Schedule at study dates. *In case of suspected hypoglycaemia MMST will be performed and capillary blood glucose level determined.

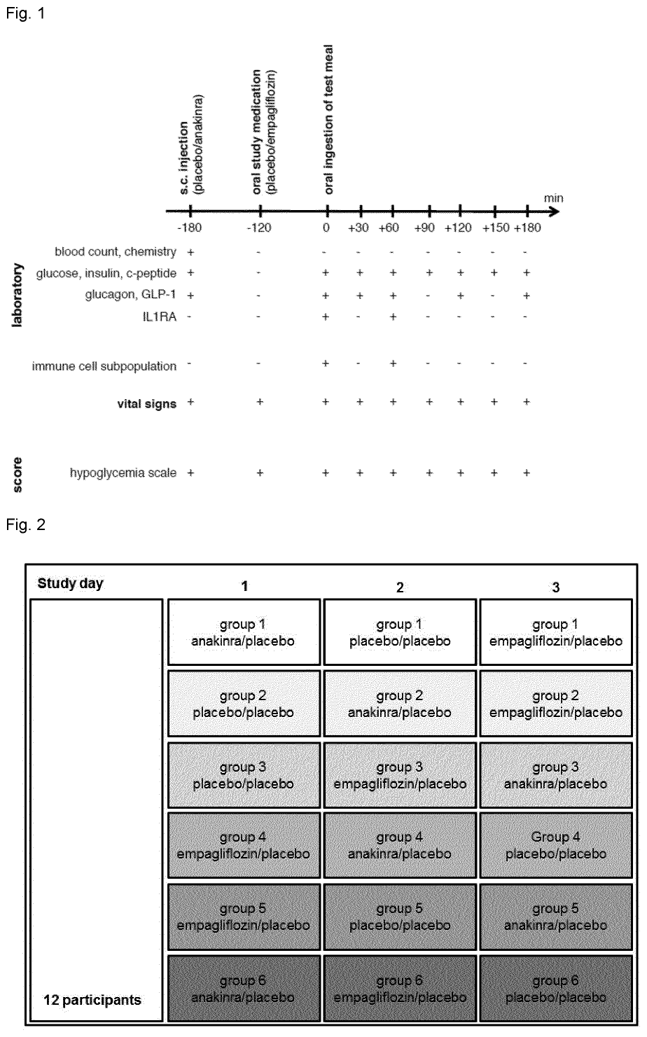

FIG. 2 Study design.

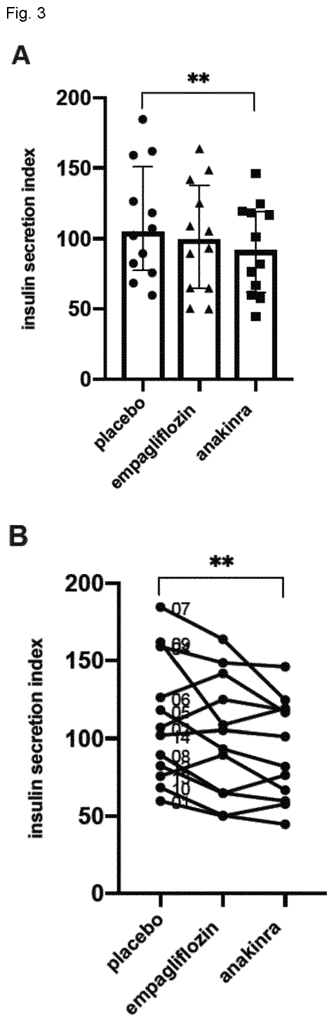

FIG. 3 A) Comparison of insulin secretion index between control and interventions. Given are median and interquartile range. Insulin secretion index was lower for empagliflozin (99.2, p-value 0.09) and significantly lower for anakinra (91.6, p-value 0.0068) compared to placebo (104.6). Level of significance was determined by using Wilcoxon-matched pairs signed rank test for comparisons between placebo and each intervention. Friedmann test for multiple group comparison was also significant (p-value 0.0087). B) Individual profiles of all subjects of insulin secretion index compared to placebo and each intervention. n=12.

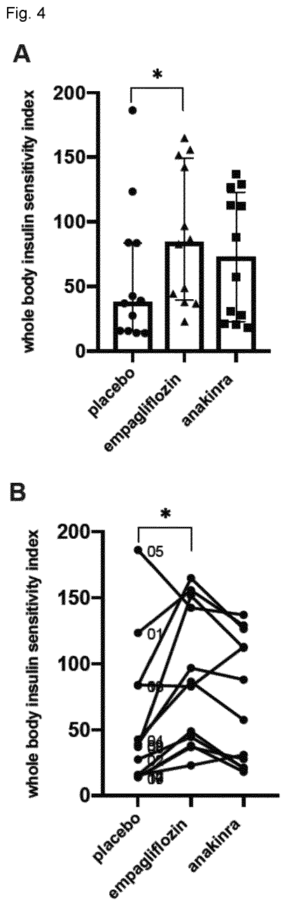

FIG. 4 A) Comparison of whole-body insulin sensitivity index between control and interventions. Given are median and interquartile range. Whole body insulin sensitivity index was significantly higher for empagliflozin (84.6, p-value 0.021) and not significantly higher for anakinra (72.1, p-value 0.064) compared to placebo (38.0) using Wilcoxon-matched pairs signed rank test for comparisons between placebo and each intervention. Friedmann test for multiple group comparison was also significant (p-value 0.0048). B) Individual profiles of all subjects of whole-body insulin sensitivity index compared to placebo and each intervention. n=12.

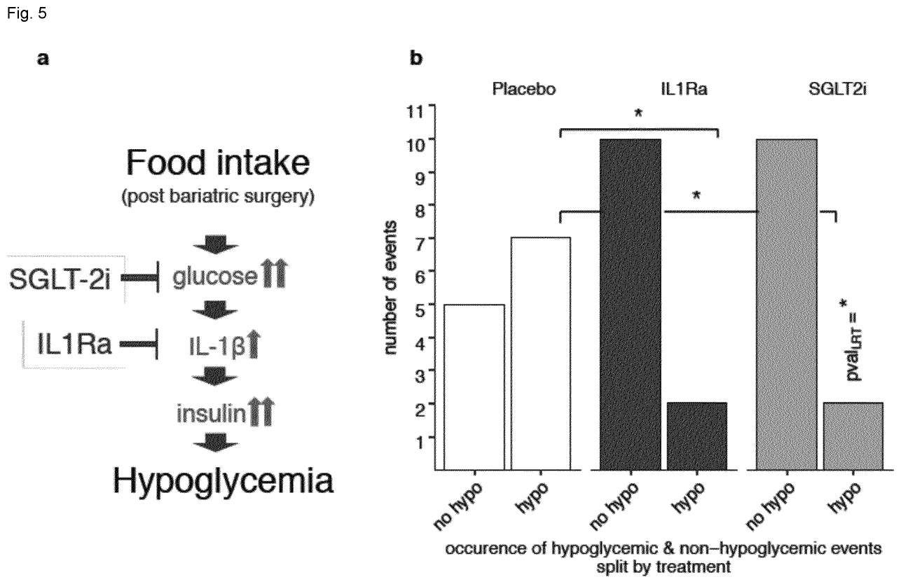

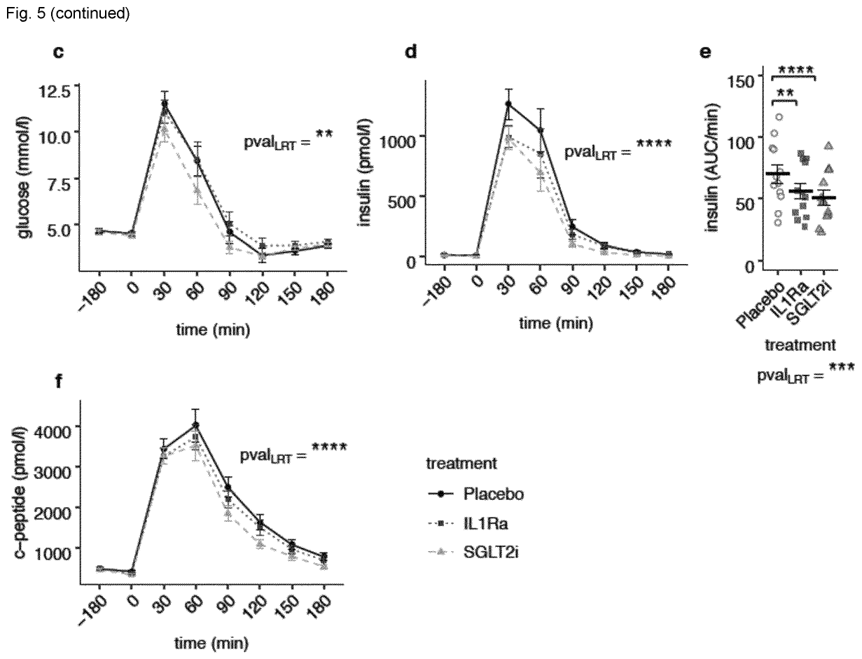

FIG. 5 Metabolic parameters in patients after bariatric surgery undergoing a mixed meal test upon treatment with empagliflozin or anakinra. [a] graphical abstract summarizing the paper's main hypothesis. [b] Total number of visits without (no hypo) and with hypoglycemic (hypo) events, split according to treatment condition. Plasma glucose [c] and insulin [d] with corresponding area under the curve (AUC) [e] and c-peptide levels [f]. Of note, placebo-treated patients required more often rescue glucose administration, therefore glucose levels after 60 minutes are artificially increased compared to the empagliflozin- and anakinra-treated groups. Data are presented as arithmetic mean (horizontal bars in e and points in time-series analysis=c, d and f) ±SD (errorbars). Statistical Analysis was performed using linear and generalized linear additive mixed-effect models as described in the methods section unless otherwise indicated (pval_{LRT}=overall p-values generated by Likelihood Ratio Test comparison of full vs. reduced model). P-values for individual comparisons were obtained by a subsequent Benjamini and Hochberg posttest for multiplicity adjustment. For details regarding LRT-test results and effect size estimates see Table 6; *p<0.05, **p<0.01, ***p<0.001, ****p<0.0001.

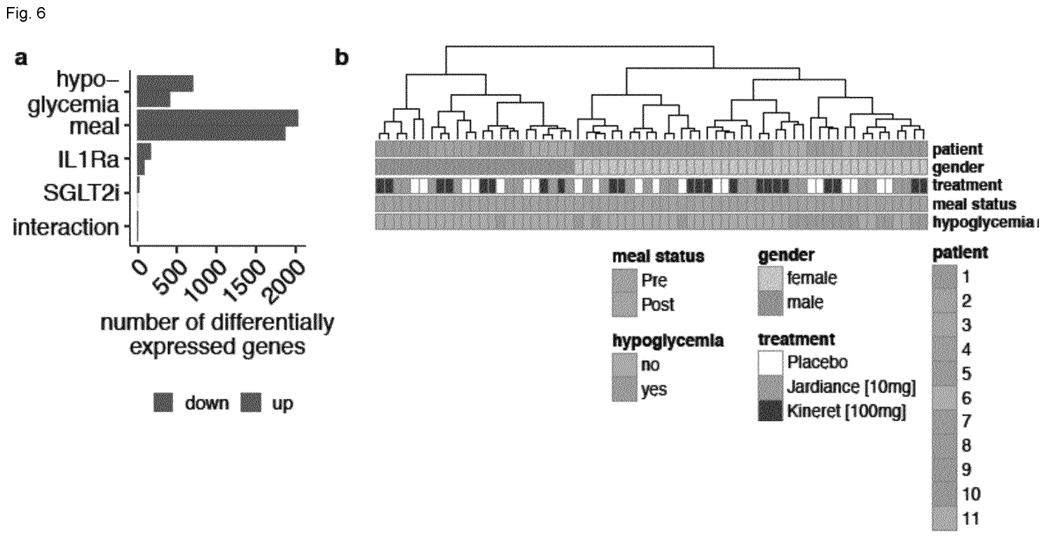

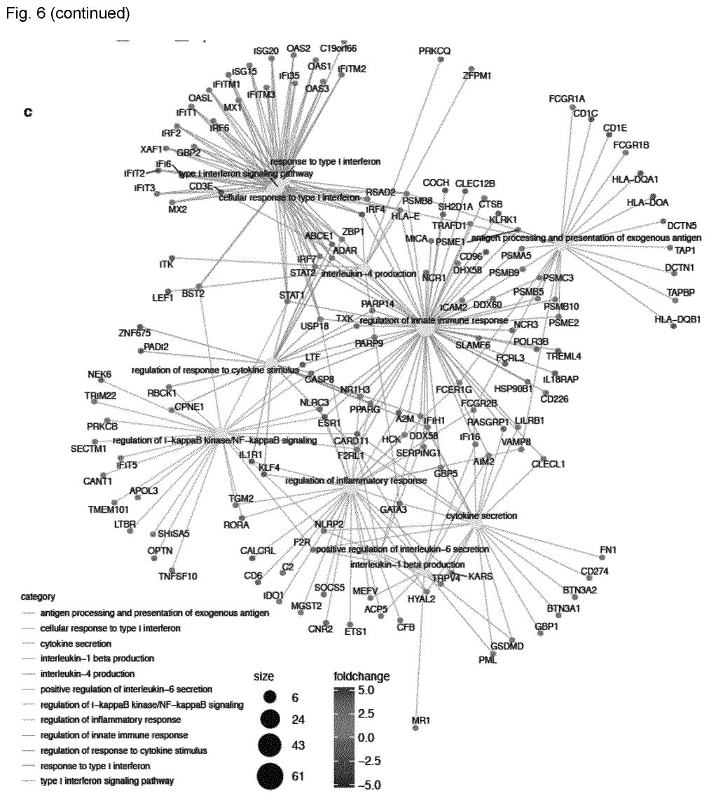

FIG. 6 Monocytes taken from patients on days with hypoglycemia show upregulation of inflammatory pathways. [a] Number of genes differentially expressed (up or down-regulated) in patients presenting with hypoglycemia, between patient samples pre- and post-meal (meal), and samples treated with anakinra (IL1Ra) and empagliflozin (SGLT2i). [b] Dendrogram produced by unsupervised hierarchical clustering of the 20 most highly variably expressed genes across all sequenced samples. Patient, gender, treatment, pre- or post-meal and hypoglycemia for each sample is indicated in the colored squares below the dendrogram [c] Gene-Concept Network plot (cnet plot) of significantly enriched GO-pathway terms and corresponding differentially expressed up- (red) or downregulated (blue) genes in the comparison of hypoglycemia with no hypoglycemia.

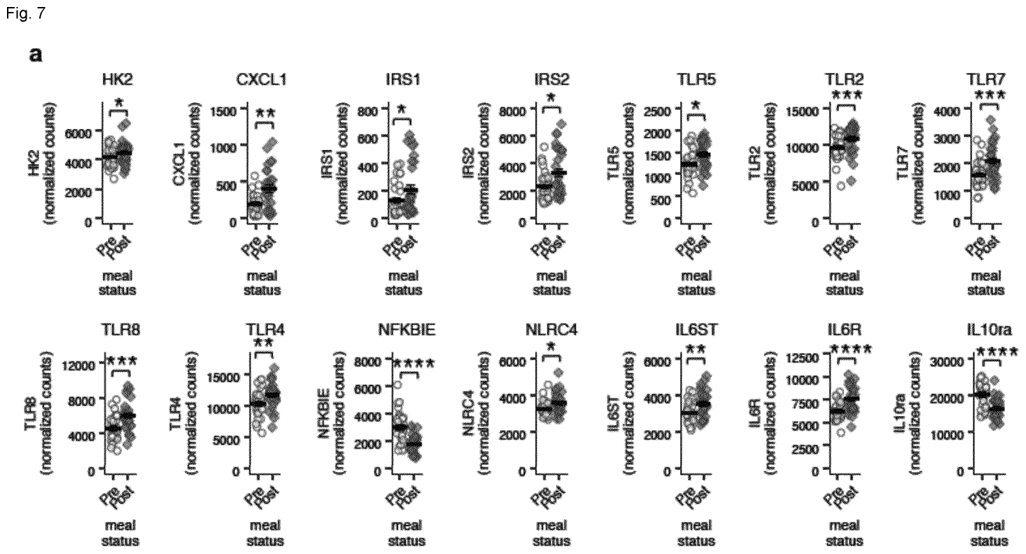

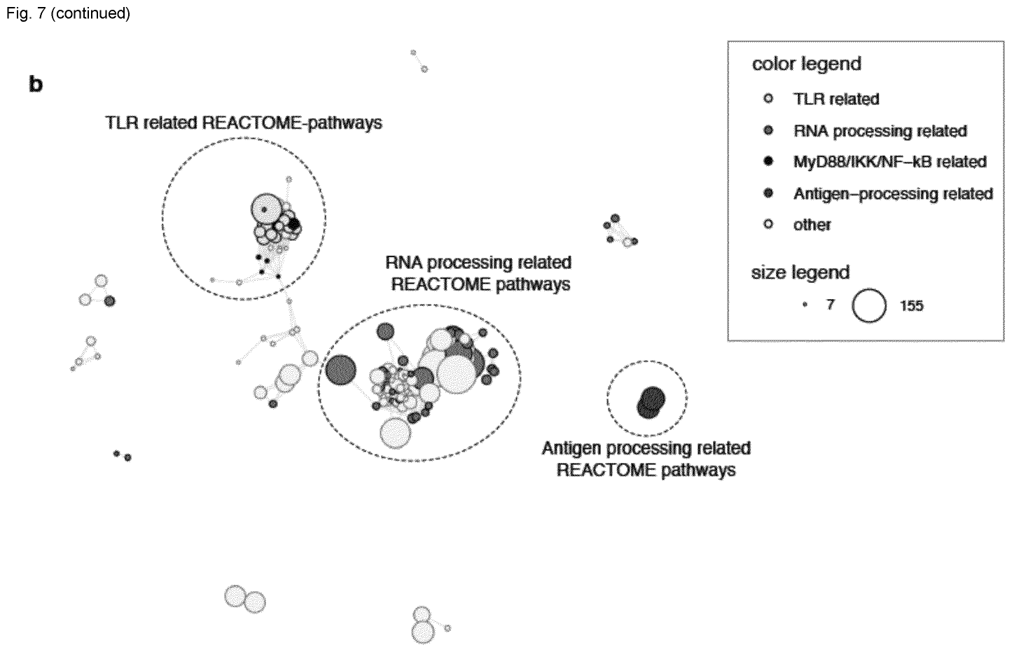

FIG. 7 Food-intake induces gene expression of pattern-recognition receptors, as well as immune and metabolic pathways in patients post-bariatric surgery. [a] Selection of differentially expressed genes in monocytes isolated from patients post-bariatric surgery before (pre) and after (post) a mixed meal test. Data are presented as overall arithmetic mean of normalized count data for each gene (horizontal bars) ±SD (errorbars). [b] Network of all significantly enriched REACTOME-pathway terms in monocytes isolated from patients post-bariatric surgery in the comparison before (pre) and after (post) a mixed-meal test. Individual terms are represented as single points. Distance and line connections between points indicate more (closer/more connections) or less (farther/less connection) shared genes contributing to the pathway. Closely related terms are grouped by color e.g. yellow for TLR-related pathways, red for RNA processing related ones. Point size represents the number of differentially expressed genes contributing to the pathway. Selected clusters of related terms are encircled. Statistical Analysis was performed using the R package “DESeq2” as described in the methods section. P-values for individual comparisons represent multiplicity-adjusted FDR values as implemented in the DESeq2 package (Benjamini and Hochberg posttest for multiplicity adjustment). *p<0.05, **p<0.01, ***p<0.001, ****p<0.0001.

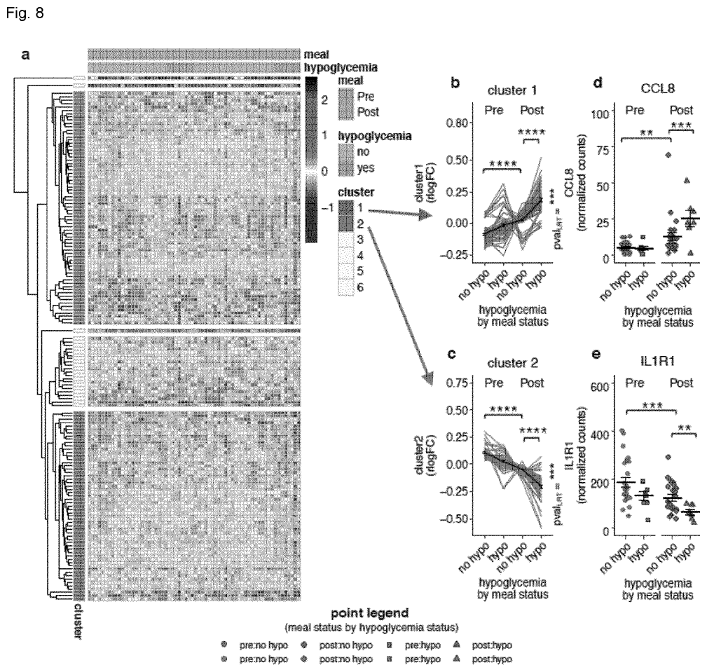

FIG. 8 Gene expression in response to hypoglycemia appears to be an exaggeration of the response to food-intake. [a] Heatmap of differentially expressed genes overlapping in the gene expression-level in response to hypoglycemia and to food-intake. Displays regularized log-fold changes from base mean and the result of unsupervised hierarchical clustering, separating these genes into two large clusters (1 and 2). [b,c] Analysis of the mean trend in gene expression within cluster 1 [b] and 2 [c]. Displayed are plots of regularized log-fold change from base mean for each gene found in both the comparison hypoglycemia (hypo)-no hypoglycemia (no hypo) and before and after food-intake. Plots displaying normalized counts of two representative genes in clusters 1 [d] and 2 [e]. Data are presented as gene-specific arithmetic mean (individual colored lines in plots c (red) and e (blue)) and overall arithmetic mean (black lines in plots b and d, horizontal bars in plots d and e) ±SD (errorbars). Statistical analysis was performed using linear mixed-effect models as described in the methods section (pval_{LRT}=overall p-values generated by Likelihood Ratio Test comparison of full vs. reduced model). P-values for individual comparisons were obtained by a subsequent Benjamini and Hochberg posttest for multiplicity adjustment. For details regarding LRT-test results and effect size estimates see Table 6; *p<0.05, **p<0.01, ***p<0.001, ****p<0.0001.

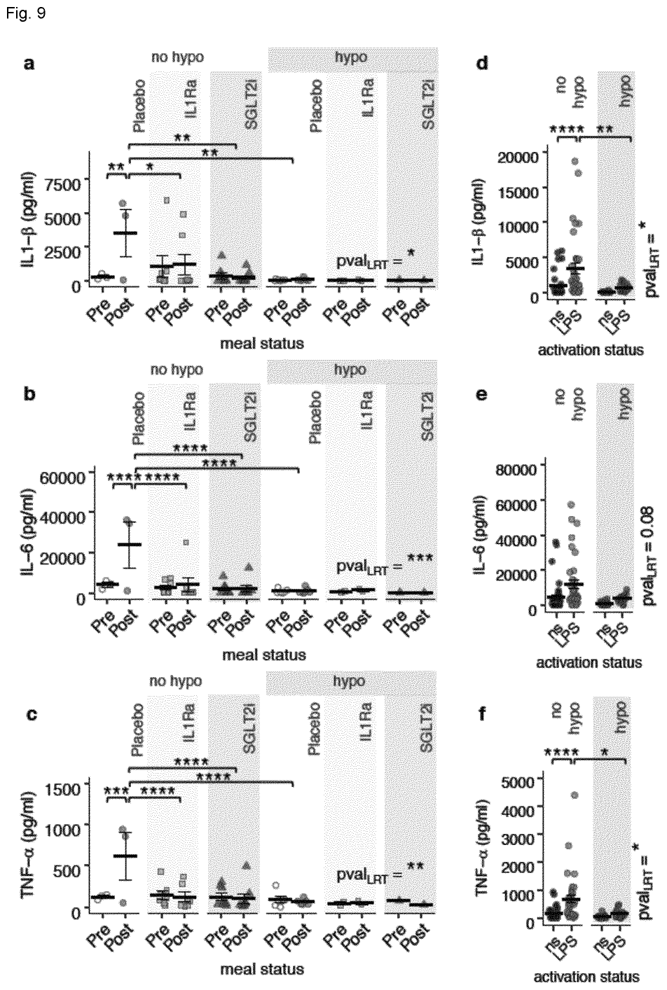

FIG. 9 Ex vivo cytokine secretion in meal- and hypoglycemia-preconditioned monocytes. Protein measurements of IL-1β [a], IL-6 [b] and TNF-α [c] in cell supernatants taken from non-stimulated bulk monocytes isolated from patients that subsequently developed hypoglycemia (hypo) or not (no hypo), and treated with either placebo, anakinra or empagliflozin, pre- and post-liquid mixed-meal test. Protein measurements of IL-1β [d], IL-6 [e] and TNF-α [f] in cell supernatants taken from bulk monocytes isolated from patients post-bariatric surgery that either did (hypo) or did not (ho hypo) respond with hypoglycemia to a mixed-meal test. Cells were isolated and subsequently stimulated with LPS or left unstimulated (ns=non-stimulated condition, LPS=LPS stimulated condition). Data are presented as arithmetic mean (horizontal) ±SD (errorbars). Statistical analysis was performed using linear mixed-effect models as described in the methods section (pval_{LRT}=overall pvalues generated by Likelihood Ratio Test comparison of full vs. reduced model). P-values for individual comparisons were obtained by a subsequent Benjamini and Hochberg posttest for multiplicity adjustment. For details regarding LRT-test results and effect size estimates see Table 6; *p<0.05, **p<0.01, ***p<0.001, ****p<0.0001.

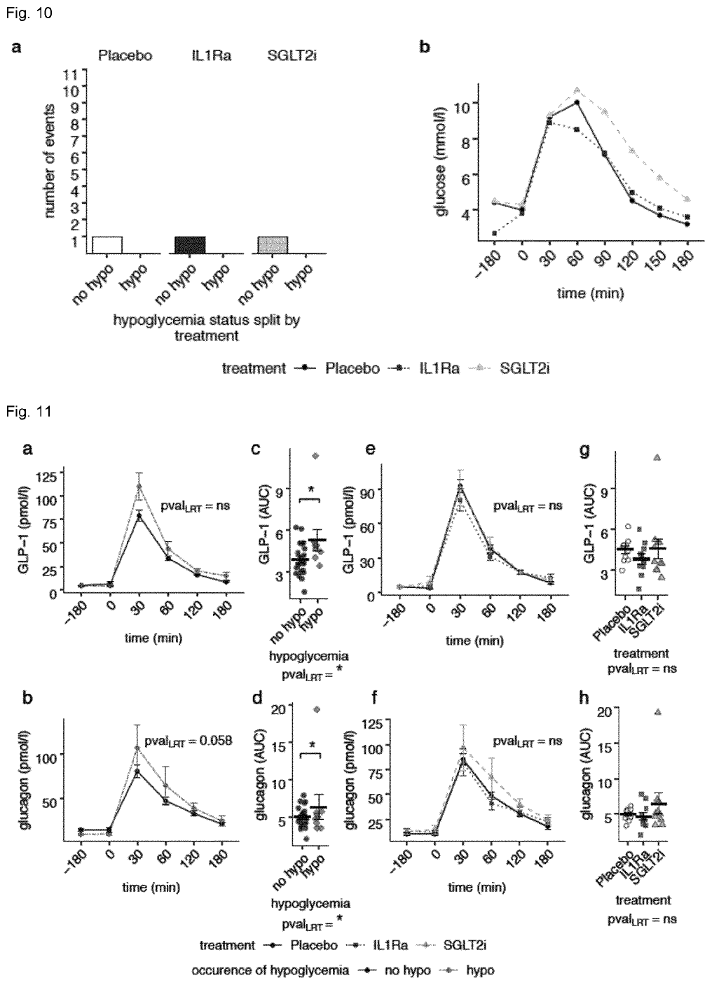

FIG. 10 Hypoglycemic events and plasma glucose and for patient 3. [a] Total number of visits without (no hypo) and with hypoglycemic (hypo) events, split according to treatment condition. [b] Plasma glucose over time. Data are presented as number of events (colored bars in a) or individual values over time (colored lines in b). Circulating insulin, c-peptide, GLP-1 and glucagon levels were not measured for this patient.

FIG. 11 Prevention of hypoglycemia by empagliflozin or anakinra is independent of changes in GLP-1 or glucagon: circulating GLP-1 [a] and glucagon [b] in patients presenting with hypoglycemia (=hypo) or not (=no hypo). Corresponding area under the curve (AUC) estimates for GLP-1 [c] and glucagon [d]. Circulating GLP-1 [e] and glucagon [f] by treatment condition. Corresponding area under the curve (AUC) estimates for GLP-1 [g] and glucagon [h]. Due to the presence of an extreme outlier in both datasets (distance from glucagon value data mean=5.149941 SD), the inventors performed sensitivity analysis for the comparison hypo-no hypo after exclusion of said outlier, results after exclusion were no longer significant (see supplementary Table S3). Data are presented as arithmetic mean (points in time-series analysis, horizontal bars in dotplots) ±SD (errorbars). Statistical analysis was performed using linear mixed-effect models as described in the methods section (pval_{LRT}=overall p-values generated by Likelihood Ratio Test comparison of full vs. reduced model). P-values for individual comparisons were obtained by a subsequent Benjamini and Hochberg posttest for multiplicity adjustment. For details regarding LRT-test results and effect size estimates see Table 6; *p<0.05, **p<0.01, ***p<0.001, ****p<0.0001.

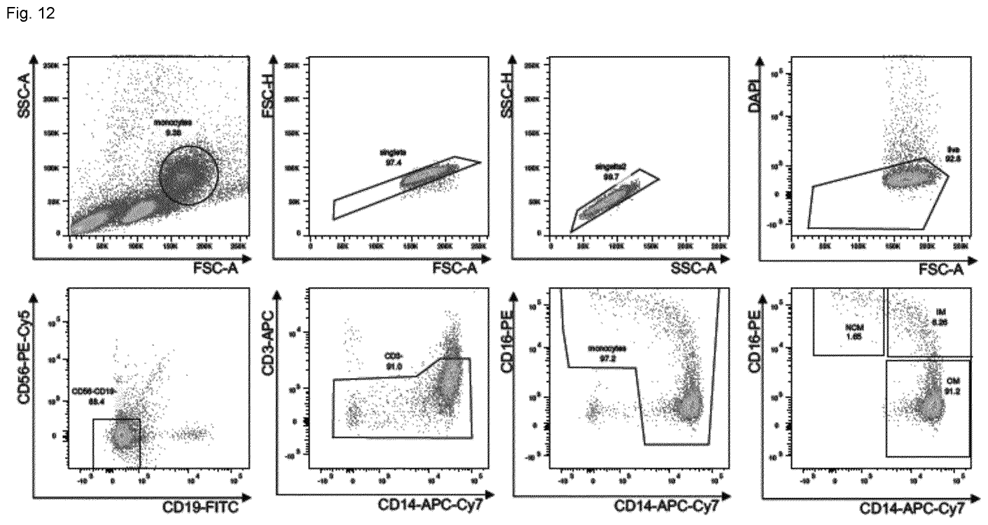

FIG. 12 Representative Plots detailing the sorting strategy employed for the isolation of bulk monocytes. Upper Panel: Graphs detailing gating for Forward-Sideward Scatter, single cell discrimination and live-cell staining. Lower panel: First two graphs from the left detailing elimination of cells positive for lineage markers CD3, CD19 or CD56. Third graph from the left, final gate for the bulk monocyte population. Final graph from the left, gates for monocyte subset isolation [non-classical monocytes (NCM), intermediate monocytes (IM), classical monocytes (CM)].

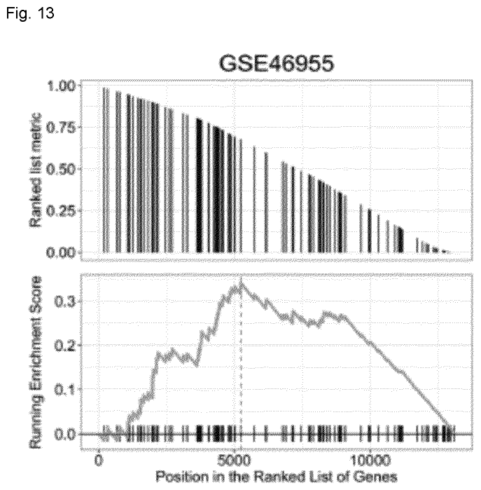

FIG. 13 GSEA enrichment analysis of an “Endotoxin Tolerant Gene Signature” in hypoglycemic patients. Plot of Gene Set Enrichment Analysis, showing enrichment of an “Endotoxin-Tolerant Phenotype” (taken from publicly available dataset GSE46955) in monocytes taken from patients with symptomatic hypoglycemia as compared to controls (p=0.00016, Fold Enrichment=19.7). Statistical analysis was done using the R package “pathfindeR”.

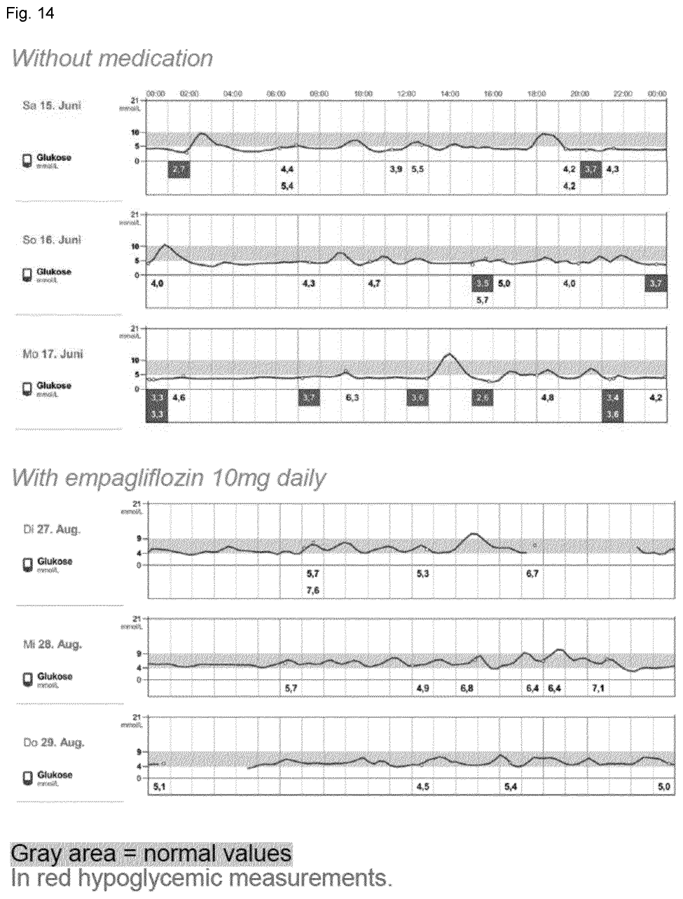

FIG. 14 Representative continuous glucose monitoring report of a 31-year old female patient before and during treatment with 10 mg empagliflozin daily. Gray shaded areas represent glucose values within normal range. Red colour indicates time spent in hypoglycemia.

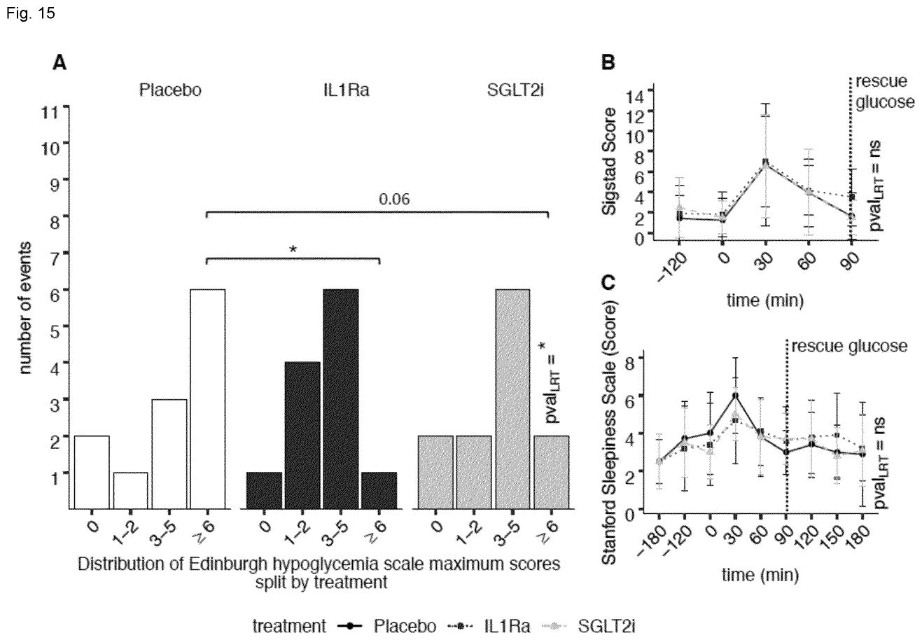

FIG. 15 Edinburgh Hypoglycemia Scale, Sigstad Score and Standford Sleepiness Scale Score by treatment. [a] Distribution of total number of visits, split by maximum Edinburgh Hypoglycemia Score and respective treatment condition. Sigstad Scores [b] and Stanford Sleepiness Scale Scores [c] over time and split by treatment. Data are presented as number of events (colored bars in a) or as arithmetic mean (points in time-series analysis=b and c) ±SD (error bars). Vertical lines in b and c indicate the border between timepoints that were free of confounding (to the left of the line) and those that were confounded by unequal rescue glucose administration to the different treatment groups (to the right of the line). Statistical Analysis was performed using linear and generalized additive linear mixed-effect models as described in the methods section unless otherwise indicated (pval_{LRT}=overall p-values generated by Likelihood Ratio Test comparison of full vs. reduced model). For details regarding LRT-test results and effect size estimates see supplementary Table S4; *p<0.05.

### EXAMPLES

Patients are eligible for this study when they underwent bariatric surgery and have proven post-prandial hypoglycaemia either with random post-prandial plasma glucose levels, continuous glucose monitoring or within a mixed-meal test. Patients will participate in the study after thorough information and providing written informed consent.

### Example 1: General Study Design and Justification of Design

This study is a placebo controlled, double-blind, randomized, cross-over proof-of-concept study. The study consists of a screening visit and three study dates that need to have a minimum interval between the dates of four days and maximum of two weeks. Ideally, all three study dates are performed within 14 days allowing the usage of one single flash glucose monitoring system sensor (Freestyle Libre®, Abbott) for the entire period. The screening visit is about 90 min. Each study day lasts 6.5 hours and consists of the same schedule. If the subject agrees to use a glucose flash monitoring system, the sensor is installed the afternoon before study day 1 and the subject is instructed in the use of the system.

The 12 subjects were randomly assigned into six groups consisting of two subjects receiving study medication in a double-blind, crossed-over manner as outlined in FIG. 2. Patients need to be sober at least eight hours before ingestion of mixed-meal. To ensure that fasting period is not too long, patients receive a small late evening meal consisting of 4 DarVida Natural Crackers as well as 3 Sinergy® drops (see Appendix for specific contents) or are alternatively pleased to have their dinner at around 10 pm. Treatment consists of a single subcutaneous injection of 100 mg Anakinra (Kineret®) or matched placebo three hours before ingestion of the mixed-meal and of a single oral tablet of 10 mg of empagliflozin (Jardiance®) or matched placebo two hours before mixed-meal at each study day. Patients were randomised to receive in different sequences either (1) anakinra and an oral placebo to empagliflozin, (2) empagliflozin and a subcutaneous placebo (0.67 ml 0.9% saline) to anakinra or (3) double placebo. Participants, investigators and study nurses were blinded to the study drug.

Between the study drug application questionnaires concerning dumping symptoms were answered. Regular measurement of vital parameters, blood samples and hypoglycaemia assessments were performed as outlined in FIG. 1. After ingestion of the liquid mixed-meal subjects had to rest in supine position (at least 45°). One subject was not able to ingest the full specified liquid mixed meal (carbohydrate load of 40 g instead of 60 g) on the first study day and thereafter received the same portion of liquid mixed meal on subsequent study days.

Due to practical reasons or due to preventive interventions to comply with defined timepoints absolute timepoints may deviate in average 5+/−5 minutes from the defined ones, hence this issue will be further addressed in the chapter of the limitations of this study. In case of symptomatic hypoglycaemia before the prespecified next laboratory control immediate glucose measurements with Freestyle Libre® and POCT respectively were performed and in case of hypoglycemic values (<3.0 mmol/l) laboratory samples were drawn and glucose (10 g) was given intravenously. Samples were named according to the exact timepoint and the following pre-specified samples were taken according to the study schedule. If patients were not symptomatic glucose (10 g) was given intravenously only in case of POCT glucose values below 2.5 mmol/l.

### Example 2: Methods of Minimising Bias

The study was placebo controlled, double-blinded, randomized and crossover to minimise bias.

Randomization

After the screening procedures, subjects were randomized to either group 1-6 (ratio 1:1, in blocks of 2 participants).

Group 1 received anakinra on the first study day, and the next study day only placebo and empagliflozin on the third study day. Group 2 received only placebo on the first study day, on the second anakinra and on the third study day empagliflozin and so on as outlined in FIG. 1.

Subjects were assigned a unique subject identification number according to the randomization schedule. Randomization was performed by an independent, qualified scientist at the University Hospital Basel. Randomization log were handed over to the independent study nurse. Randomization was done blockwise to ensure equal distribution of investigational drug use at the study dates for the total of 12 participants. In case of drop-outs or screen failures adjustment by an independent qualified scientist was performed to ensure equal interventions at study dates.

Blinding Procedures

Both subjects and investigators were blinded. When all subjects had completed final visit assessments, the data were cleaned and locked, and personnel was unblinded in order to complete analysis of data. A nurse independent of the research group was responsible for treatment blinding and preparation of trial drugs throughout the study.

All active and placebo vials were randomized with a kit number. Subjects were assigned a unique subject ID and kit number according to the randomization schedule. The label on the vial indicated the study, the subject ID and also the kit number, but did not indicate the treatment assignment.

Unblinding Procedures (Code Break)

After all subjects had completed the study, the data was unblinded to the investigators in order to complete analysis of data. Drop outs were replaced.

Unblinding of individual treatment assignment occurred when medically necessary. For each study participant a closed envelope containing the study intervention assignment was placed in a specified room of the clinic of endocrinology at the university hospital Basel accessible for the doctor on call at the endocrinology division.

### Example 3: Study Population

Eligibility Criteria

Inclusion criteria:
Patients after bariatric operation (i. e. roux-y-gastric bypass or biliopancreatic diversion) with documented hypoglycemia, i. e. ≤2.5 mmol/l and hypoglycaemic symptoms.
Age ≥18 years
For subjects with reproductive potential, willingness to use contraceptive measures adequate to prevent the subject or the subject's partner from becoming pregnant during the study

Exclusion Criteria:
Signs of current infection
Use of any investigational drug in the last four weeks prior to enrolment (Anakinra, Empagliflozin)
Use of any anti-diabetic drugs
adrenal insufficiency and/or substitution with glucocorticoids
Neutropenia (leukocyte count<1.5×109/L or ANC<0.5×109/L)
Anemia (hemoglobin<11 g/dL for males, <10 g/dL for females)
Clinically significant kidney or liver disease (creatinine>1.5 mg/dL, AST/ALT>2×ULN, alkaline phosphatase>2×ULN, or total bilirubin [tBili]>1.5×ULN)
Current immunosuppressive treatment or documented immunodeficiency
Uncontrolled congestive heart failure
Uncontrolled malignant disease
Currently pregnant or breastfeeding
Intolerance to lactose.

Recruitment and Screening

Patients were recruited and screened at the department of diabetology and the medical outpatient department at the University Hospital Basel. Patients were also asked if they are interested to be contacted to get information about the study within the endocrine network clinics at cantonal hospital Lucerne and Aarau as well as St. Claraspital Basel, all Switzerland.

### Example 4: Experimental Intervention

Experimental Intervention (Treatment)

Empagliflozin may be administered as the white round tablet currently available in pharmacies. Each tablet contains the active substance empagliflozin as well as the adjuvant lactose-monohydrate.

Anakinra (Kineret®; r-metHuIL-1ra, Swedish Orphan Biovitrum AB) is a recombinant, non-glycosylated form of the human interleukin-1 receptor antagonist (IL-1Ra) in a 100 mg/0.67 ml solution for subcutaneous injection. It differs from native human IL-1 Ra in that way that it has the addition of a single methionine residue at its amino terminus. Kineret® consists of 153 amino acids and has a molecular weight of 17.3 kilodaltons and is produced by recombinant DNA technology using an E. coli bacterial expression system.

Mild and transient injection reactions might occur in 20-50% after 2-4 weeks of injections. In the present study, only a single injection will be applied and therefore injection site reactions are not expected.

Anakinra is FDA approved for the treatment of rheumatoid arthritis in the US as well as in Europe and has an acceptable risk/benefit profile in this indication, with more than 100.000 patients treated. Most common adverse events include mild and transient local injection reactions in 20-50% of subjects treated with Anakinra. Consistent with its mechanism of action, Anakinra reduces WBC/ANC in 2.4% of patients and this may increase the risk of infection. Accordingly, treatment with Anakinra will not be initiated in patients with active infections. Safety will be monitored by physical exams and blood tests.

Control Intervention (Comparator Treatment)

Subcutaneous and per oral administration in physically blinded participant via an independent study nurse.

Subcutaneous injection was sterile 0.9% saline solution. As oral placebo control Winthrop P® (Zentiva, Frankfurt/Main) lactose tablet was used.

Experimental Intervention

Empagliflozin (Jardiance)® 10 mg tablet was taken on one of the three study days. On the other study days subjects received placebo two hours before ingestion of the mixed-meal.

Subjects received an s.c. injection of 100 mg Anakinra on one of the three study days, on the other study days subjects received placebo three hours before ingestion of the mixed-meal.

Control Intervention

Placebo tablet was taken on two study days two hours before ingestion of mixed-meal.

s.c. injection of saline was taken on two study days three hours before ingestion of mixed-meal.

Dose/Device Modifications

No dose or device modifications were planned for the single administration of the medications.

Data Collection and Follow-Up for Withdrawn Participants

Subjects who discontinue participation were replaced. No follow-up or additional procedures were performed on subjects discontinuing the study.

The reason(s) for a subject's discontinuation from the study must be clearly documented on the appropriate page of the CRF.

Trial Specific Preventive Measures

During the three study days regular clinical screenings and capillary glucose measurements were performed.

Blood glucose measurements regularly took place and an inserted catheter in case of hypoglycaemia was adjusted at the beginning of each study date. In case of a symptomatic decrease of blood glucose levels in symptomatic patients below 2.5 mmol/l, counter-regulatory actions (e.g. 100 ml of 10% glucose) were initiated after taking the blood sample panel.

A pregnancy test was conducted in all female participants in reproductive age to rule out pregnancy prior to study start. Appropriate contraception for this study comprises using of condoms and either intrauterine devices or 3-monthly contraceptive injection or birth-control pill.

Concomitant Interventions (Treatments)

No changes of the ongoing medical therapy was made. Medication that is suspected to interfere in any way must not be used (s. exclusion criteria). The used medication was recorded in the CRF.

Study Drug/Medical Device Accountability

From shipment to the site until return or disposal, study drugs were accurately and adequately monitored. Dates of receipt/expiry/use/return were recorded.

### Example 5: Study Assessments

Assessment of Primary Outcome

The primary outcome in this study is the change of post-prandial hypoglycaemia after treatment with empagliflozin (Jardiance®) or anakinra (Kineret®) compared to placebo. This was assessed by blood glucose measurements (capillary POCT measurements and plasma glucose) as well as clinical scoring systems (Edinburgh Hypoglycaemia Scale (Deary et al. Diabetologia 1993; 36(8): 771-7), Mixed-Meal-Test (Wiesli et al. Eur J Endocrinol 2005; 152(4): 605-10.) Stanford sleepiness scale).

Assessment of Secondary Outcomes

The secondary objectives are to evaluate whether treatment of empagliflozin (Jardiance®) or anakinra (Kineret®) have effects on the following aspects:
level of insulin secretion and sensitivity
levels of GLP-1, glucagon and C-peptide
levels of IL-6; IL-1Ra, CRP
scores in Edinburgh Hypoglycaemia Scale (clinical score for assessment of hypoglycemia), Stanford Sleepiness Scale (clinical score of assessment of sleepiness) as well as Sigstad Score (clinical score for early and late dumping symptoms)
Length of time and amount of glucose needed for restoring normoglycemia.

Assessment of Safety Outcomes

Adverse Events

During the entire study, i. e. study day 1 until end of study day 3 all adverse events (AE) and all serious adverse events (SAEs) were recorded and fully investigated based on observed or voluntarily reported signs and symptoms as well as findings in the participant's physical examination and/or laboratory results. All AEs of category Ill were documented according to Common Terminology Criteriar for Adverse Eventes (CTCAE v4.0) on the respective AE pages of the CRF.

Medical events that occur from the time the subject signs the informed consent form to the time the administration of the first study day were not considered as AEs, and were recorded under medical history.

Laboratory Parameters

Within this study repeatedly, varying tests were performed. If clinically significant abnormal laboratory test values are noticed that are unexpected, the test was repeated and the subject was followed until the test value has returned to normal range or the investigator has determined that the abnormality is chronic or stable. Every pathologic laboratory finding was evaluated of its clinical relevance. An isolated abnormal laboratory result in the absence of further associated clinical findings may or may not be considered as an AE. An abnormal laboratory result was considered clinically relevant as an AE when it is part of a clinical abnormality requiring specific medical intervention or follow-up. Since the effect on hypoglycaemia is the primary endpoint, low blood glucose level was not reported as AE.

Vital Signs

Blood pressure and heart frequency measurements were performed regularly as well as scores concerning neurocognitive function (see FIG. 1).

### Example 6: Procedures at Each Visit

Screening Visit, Day −60 to −7
1. All subjects were checked for eligibility criteria. If all eligibility criteria were fulfilled, participants were requested to sign the informed consent.
2. A standardized medical history questionnaire and concomitant therapy/Intervention questionnaire was filled out.
3. A physical examination was performed and vital signs (heart rate, blood pressure, body temperature, weight, height) were recorded.
4. Blood sample.

Study Day (0—Optional)

If patients agreed to use the flash glucose monitoring system (Freestyle Libre®, Abbott) the sensor was placed on the back of the upper arm and patients were instructed in using the detector.

Study Day (1-3)

At each study day the same schedule took place. After a routine physical examination and baseline diagnostics patients were randomized, blinded and receive the subcutaneous study medication (placebo/anakinra) three hours before the mixed-meal test by an independent study nurse. Afterwards various questionnaires were performed. The tablet (placebo/empagliflozin) was administered by an independent study nurse two hours before the mixed-meal test. After ingestion of the mixed meal test regular measurements of vital signs, glucose level, blood samples were performed as well as clinical testing for neurocognitive and vegetative signs of hypoglycaemia for three hours after ingestion of mixed-meal.

### Example 7: Insulin Secretion

Insulin secretion was measured using standard insulin ELISA. Insulin secretion index was lower for empagliflozin (99.2, p-value 0.09) and significantly lower for anakinra (91.6, p-value 0.0068) compared to placebo (FIG. 3).

Whole body insulin sensitivity index was significantly higher for empagliflozin (84.6, p-value 0.021) and not significantly higher for anakinra (72.1, p-value 0.064) compared to placebo (38.0) using Wilcoxon-matched pairs signed rank test for comparisons between placebo and each intervention (FIG. 4).

Empagliflozin reduced peak glycaemia at 30 (11.2 vs. 10.1 mmol/l), 60 (9.1 vs. 6.9 mmol/l) and 90 (4.5 vs. 3.5) minutes after ingestion of the mixed meal compared to placebo and was followed by a significant reduction of glucose-requiring hypoglycaemic events (n=2, 16.6%) compared to placebo (n=8, 61.5%). In contrast, treatment with Anakinra did not result in significant changes of the glucose curve within the first 90 minutes, but was also followed by a significantly reduced rate of glucose-requiring hypoglycaemic events (n=2, 16.6%) compared to placebo (n=8, 61.5%). Both treatment interventions showed significantly lowered insulin secretion compared to placebo.

Individual body weights remained stable throughout the study period. On days where patients received an injection of anakinra, serum levels of IL-1Ra increased from an average of 272 pg/ml (95% CI=244.16-299.19) to 43836 pg/ml (95% CI=43707-43965), confirming correct application and resorption of the drug. Leukocyte and CRP levels were not elevated and remained stable throughout the trial.

On the day of placebo administration, one to three hours following the ingestion of the mixed meal, 7 of 12 patients developed severe symptomatic hypoglycemia requiring glucose administration (FIG. 5a). All patients responded to 10 g glucose administration with immediate and complete resolution of their hypoglycemic symptoms, thus fulfilling the Whipple's triad. One patient on placebo developed another hypoglycemic episode after blood glucose normalization that again resolved immediately after administration of an additional 10 g of glucose (Table 3). In contrast, pre-treatment with either anakinra (p=0.037) or empagliflozin (p=0.037) significantly reduced the severity of hypoglycemic events with only two events requiring rescue glucose administration (FIG. 5a). The reduction of the severity of the symptoms by treatment was also documented on the Edinburgh Hypoglycemia Scale (FIG. 15a, and Table 4). Moreover, empagliflozin reduced peak postprandial glucose levels (FIG. 5b). Of note, the majority of placebo-treated patients required rescue glucose administration, therefore, glucose levels at later time points (>60 minutes), i.e. following rescue glucose administration, are artificially increased in the placebo-treated group as compared to the empagliflozin- and anakinra-treated groups.
Anakinra and empagliflozin treatment reduced insulin release (FIG. 5c,d), as well as c-peptide secretion over time (FIG. 5e) most likely thereby preventing hypoglycemic events. Furthermore, anakinra reduced the insulin secretion index (p=0.005), while empagliflozin showed a trend in same direction (p=0.08) [placebo average 108.0 pmol/mmol (95% CI=84.4 to 132); effect estimate for anakinra −18.5 pmol/mmol (95% CI=−29.3 to −7.66); effect estimate for empagliflozin −10.8 pmol/mmol (95% CI=−21.6 to 0.0433)]. Serum GLP-1 and glucagon levels were not affected by either treatment (FIG. 11). Of note, due to the rapidity and severity of the hypoglycemic symptoms the Mini-Mental Status test did not prove feasible in practice. The Stanford Sleepiness Scale as well as Sigstad score did not show significant differences between treatments, but were equally confounded by imbalanced rescue glucose application between treatment groups (FIG. 15b,c).

Monocytes of Patients, Isolated on Days with Hypoglycemia, Display Increased Inflammatory Gene Expression.

Following the inventors' hypothesis of an overactivation of the innate immune system in patients experiencing post-bariatric hypoglycemia, they assessed transcriptional changes in innate immune cells from the inventors' patient population using an unbiased RNA sequencing approach. Therefore, the inventors isolated monocytes from peripheral blood sampled immediately before and 60 minutes (i.e. before rescue glucose application when needed) after ingestion of the mixed-meal on all three treatment days.

Samples taken during study days with hypoglycemic events differed profoundly from samples taken on study days that did not require glucose-rescue. Indeed, a total of 1111 genes were differentially regulated in this comparison, with 702 genes being upregulated and 409 downregulated (FIG. 6a). Next, the inventors performed unsupervised hierarchical clustering of the 20 most variably expressed genes across all samples. As expected, gender and patient-specific effects accounted for large differences between samples (FIG. 6b). However, whether hypoglycemia occurred during the visit or not explained more of the variance than either meal or treatment status, as indicated by clustering of these samples at the right edge of the dendrogram (FIG. 6b).

Subsequently, the inventors investigated the gene expression signature that distinguished study visits with hypoglycemic episodes from those without hypoglycemia. Gene Ontology (GO) pathway analysis of the differentially expressed genes obtained in this comparison revealed that pathways for “interleukin-1β production”, “positive regulation of interleukin-6 secretion” as well as “cytokine secretion” and “regulation of inflammatory/innate immune response/cytokine stimulus” were significantly enriched in these samples (FIG. 6c). To test the robustness of these results, the inventors additionally performed weighted gene correlation network analysis with their data set. Pathway analysis revealed “interleukin-1-mediated signaling” and “positive regulation of interleukin-1-mediated signaling” among the top 5 significantly enriched pathways in the thistle2 module significantly correlating both with whether hypoglycemia happened on a study day or not and status pre or post mixed-meal test (data not shown).

Next, the inventors assessed the effect of the meal on monocyte transcriptome. Samples taken pre-meal differed vastly from samples taken post-meal (total number of differentially regulated genes=3901, number of upregulated genes=2025, number of downregulated genes=1876). Specifically, the inventors saw marked sings of immune cell activation. Most prominently, there was a broad activation of gene-expression encoding for Toll-like receptors (TLRs). Indeed, TLR 2, 4, 5, 7 and 8 were all significantly upregulated (FIG. 7a). Likewise, pathway analysis of differentially expressed genes equally showed upregulation of various TLR cascades and MyD88 and NF-kB pathways (FIG. 7b). Similar to the inventors' previously published data in mice, they could observe upregulation of genes encoding for HK2, CXCL1, CCL2 and downstream targets of the Insulin Receptor (IRS1, IRS2) upon mixed-meal ingestion (FIG. 7b).

The inventors then compared whether the response to food-intake itself differed between days with and without hypoglycemia. An interaction analysis revealed barely any difference between the response to food-intake in samples taken on study days where hypoglycemia occurred as compared to days where it did not (number of upregulated genes=1, number of downregulated genes=2; FIG. 6a). This was confirmed by analyzing the mean trend in gene expression for genes differentially expressed in both conditions (number of overlapping genes=151). Unsupervised hierarchical clustering separated these 151 genes into 2 large clusters (accounting for 80% of overlapping genes) (FIG. 8a). Herein, cluster 1 (70/151 genes) represented genes that were significantly upregulated, while cluster 2 (51/151 genes) represented genes being downregulated in the postprandial state. Analysis of the mean trend in gene expression confirmed an average increase in gene expression from before and 60 minutes after meal ingestion in cluster 1 (FIG. 8b), as well as the respective downregulation in cluster 2 (FIG. 8c). Moreover, on average, genes in cluster 1 that were upregulated upon food-intake (representative example IRF2; FIG. 8d), were further upregulated in samples taken during visits that required glucose-rescue (FIG. 8b). Vice-versa, genes that were downregulated upon food-intake in cluster 2 (FIG. 8c), decreased even more in samples taken during visits where hypoglycemia occurred (representative example IL1-R1; FIG. 8e). Therefore, the gene signature defining a predisposition to hypoglycemia in monocytes has the characteristics of an exaggerated response to food intake.

Finally, the inventors assessed the impact of treatment with either anakinra or empagliflozin on gene expression of monocytes from their patients. Treatment with anakinra had little impact when compared to placebo (total genes differentially regulated=234, number of upregulated genes=163, number of downregulated genes=71) and empagliflozin barely changed gene expression at all (total genes differentially regulated=14, number of upregulated genes=14, number of downregulated genes=0; FIG. 6a).

Overall, these data show that on days when patients developed hypoglycemia in response to a mixed-meal test, their monocytes displayed a distinct gene expression pattern characterized by upregulated IL-1β, IL-6 and other cytokine pathways. Furthermore, ingestion of a mixed-meal equally enhanced expression of pro-inflammatory genes.

Postprandial Ex-Vivo Secretion of Inflammatory Cytokines by Monocytes is Prevented by Anakinra and Empagliflozin

In order to evaluate if changes at RNA level translated into protein expression, the inventors cultured monocytes isolated as described for RNA sequencing for a period of 18 h and measured pro-inflammatory cytokines in cell culture supernatants. Compared to monocytes isolated before food-intake, monocytes harvested post liquid mixed-meal test showed significantly increased secretion of key inflammatory cytokines including IL-1β, IL-6 and TNF-α (FIG. 9a,b,c). As expected, treatment with both, anakinra and empagliflozin, significantly inhibited this postprandial increase in inflammatory cytokine secretion as compared to placebo (FIG. 9a,b,c). Surprisingly, in samples isolated on study visits where hypoglycemia occurred, no postprandial increase in the secretion of inflammatory cytokines could be observed under any condition (FIG. 9a,b,c), potentially due to previous in vivo cytokine release. To support this hypothesis, the inventors additionally treated monocytes with lipopolysaccharide (LPS) and observed that on study days where hypoglycemia occurred monocytes failed to respond adequately to LPS stimulation (FIG. 9d,e,f). In line with the reasoning, it has been shown that monocytes taken from an inherently pro-inflammatory milieu (septic shock), express increased mRNA levels of various inflammatory cytokines and chemokines (IL-1β, IL-1α, IL-6, CCL2), but release markedly lower levels of protein following ex vivo stimulation with LPS. Accordingly, taking the publicly available microarray dataset (GSE46955) of the top 200 differentially expressed genes found in monocytes taken from patients in acute septic shock compared to those post-recovery as input, the inventors found that the “endotoxin-tolerant gene signature” was significantly enriched in monocyte samples taken on study days where hypoglycemia occurred (FIG. 13).

To summarize, monocytes taken from patients post-bariatric surgery experiencing hypoglycemia, display increased levels of pro-inflammatory gene-expression. Notably, the release of meal-induced cytokines by these same monocytes can be prevented by treatment with both anakinra and empagliflozin.

Follow Up

Two trial participants were continued to be treated, and four more patients were initiated on off-label therapy with empagliflozin after completion of the study. This led to relevant subjective improvement of their life quality and a lower frequency of hypoglycemic episodes in each of the six patients. One severely affected patient who had to ingest eight to twelve small meals (less than 20 g of carbohydrates each) a day to reduce the number of hypoglycemic episodes could significantly reduce the frequency of meals (FIG. 14). More than half a year after initiating this off-label therapy, the treatment is still effective and well tolerated by all patients.

Safety

The inventors observed no drug-related side effects following these single dose drug applications. Adverse events are outlined in Table 5.

Conclusion

Previous studies established glucose as a driver of postprandial IL-1β-induced insulin secretion in rodents. Here, the inventors could translate these observations from mouse physiology into human pathophysiology. The inventors show that both, reduction of postprandial peak-hyperglycemia with empagliflozin and blockade of glucose-induced postprandial IL-1 signaling with anakinra, reduced postprandial insulin secretion and subsequent hypoglycemia in predisposed patients after bariatric surgery. These effects were independent of changes in either glucagon or GLP-1.
Moreover, the inventors identified a dysregulation of glucose-induced postprandial inflammatory signaling as an important driver of overshooting insulin secretion in these patients. In accordance with their initial hypothesis of overactivation of the innate immune system, and specifically IL-1 signaling as a key driver of disease-pathology, the inventors' RNAseq data showed a broad upregulation of IL-1 and related cytokine pathways in monocytes taken from patients experiencing hypoglycemia.
Since food-intake can provoke episodes of hypoglycemia in patients post-bariatric surgery, the inventors equally assessed the transcriptional response to a meal. The inventors saw marked signs of immune cell activation after a mixed-meal. Genes encoding for various Toll-like receptors and subsequent inflammatory cascades were upregulated in the post-meal condition. In addition, the inventors could confirm data gathered previously in mice that showed key genes involved in metabolism and chemotaxis to be upregulated in their dataset (HK2, CXCL1, CCL2, IRS1, IRS2). Moving on to see how gene expression changes elicited by food-intake would overlap with changes seen in samples collected on study days where hypoglycemia occurred, the inventors plotted genes differentially expressed in both conditions in a heatmap of regularized log 2foldchanges. Visual inspection of this heatmap confirmed the results of the above interaction analysis. The expression level response defining a predisposition to hypoglycemia looked very similar to the one in response to food-intake. Furthermore, the mean trend in gene expression of genes that were up- or downregulated in the post-meal comparison was exaggerated in hypoglycemia, suggesting that the gene level response defining hypoglycemia in patients post-bariatric surgery might be an exaggeration of the physiologic response to food intake.
The inventors could then confirm these data at protein-level. Similar to data obtained in mice, monocytes isolated from the patients increased secretion of key inflammatory cytokines after food-intake. Interestingly, cultured cells that showed an overactivation of innate immune response pathways on a gene expression level reacted inadequately to LPS. In fact, an overactivation and subsequent inability of cultured monocytes to secrete IL-1 in other pro-inflammatory settings, like sepsis and sarcoidosis, has been described before. In line with this phenotype, the inventors found genes defining an “endotoxin tolerance signature” to be enriched in hypoglycemia.
Supporting the initial hypothesis, these data provide evidence for the presence of a pro-inflammatory state in patients with hypoglycemia after gastric bypass. Together with the fact that anakinra prevented episodes of hypoglycemia, this might indicate a reestablishment of proper physiologic immune responses in the postprandial setting.
In conclusion, the inventors provide evidence towards the presence and therapeutic importance of dysregulated pro-inflammatory signaling as the underlying cause for an overshooting insulin response in patients prone to hypoglycemia post-bariatric surgery. Above all, the inventors show that either limiting peak glycemia with empagliflozin or therapeutic modulation of the IL-1 system with anakinra can prevent hypoglycemia in these patients. Finally, the inventors provide new insights into mechanisms governing meal-induced hyperinsulinemic hypoglycemia in patients post-bariatric surgery. Importantly, the inventors offer a sound therapeutic basis for future long-term trials to base their experimental protocols on.

Empagliflozin as well as Anakinra lower glucose-requiring hypoglycemic episodes in patients after Roux-Y-gastric bypass by decreased insulin secretion. Empagliflozin may be a promising novel therapeutic option for patients with refractory postprandial hypoglycemia.

Materials and Methods

Packaging, Labelling and Supply (Re-Supply)

Empagliflozin (Jardiance®) was purchased from the pharmacy.

Anakinra (Kineret®) was purchased from Swedish Orphan Biovitrum AB, Switzerland.

Storage Conditions

Empagliflozin (Jardiance®) and lactose tablets (Winthrop P®) were kept in a secure, limited access storage area under the recommended storage conditions at room temperature between 15-25° C.

Anakinra (Kineret®) and placebo vials were kept in a secure, limited access storage area under the recommended storage conditions in the refrigerator at 2-8° C. (36-46° F.) and protected from direct sunlight until time of use. Aseptic techniques were used during withdrawal, preparation, and administration.

Appropriate forms (inventory and temperature logs) for maintenance of accurate drug accountability records were used. Used and unused study drug were stored on site until drug accountability checks had been performed by the study monitor and then be destroyed on site.

Statistical Analysis

Statistical analysis for this proof-of-concept translational clinical trial was done on a per-protocol basis. Sample size was based on practical considerations due to the lack of pre-existing data for this intervention. All analyses are to be considered exploratory. Analysis of continuous dependent outcome variables (e.g. glucose, insulin, c-peptide, GLP-1, glucagon, insulin secretion index and sensitivity, IL-1p, IL-6, IL-8, TNFα), was done using linear mixed effect models fit by maximum likelihood. Analysis of the binary dependent outcome variables occurrence of severe hypoglycemia requiring glucose rescue and occurrence of composite score ≥6 on the Edinburgh Hypoglycemia Scale was done using a generalized linear additive mixed effect model fit by maximum likelihood (Laplace approximation). Models were fit including fixed effects for categorical variables such as gender, the occurrence of hypoglycemia, treatment and in case of values measured in cell culture supernatants, activation status (non-stimulated, LPS-simulated), as well as the continuous variables time and BMI as indicated. A random-effect for patient was equally included (random by-subject intercepts, fixed slopes). Models were computed using the R “Ime4” and “gamm4” packages.
Individual models were visually assessed for the presence of heteroscedasticity, collinearity and normality of residuals. For models that did not satisfy all assumptions, outcome values were log-transformed to better approximate a gaussian distribution (insulin, c-peptide, GLP-1, glucagon) and re-fitted. For visual representation, log-transformed outcome values were back-transformed to their initial values.

Inferential statistics were done comparing fully fitted models to reduced models lacking the comparator of interest with likelihood ratio tests (LRT) using the Satterthwaite method for denominator degrees of freedom for F-tests.

In case the full model significantly better described the data, post hoc testing was done using the R “multcomp” and “emmeans” packages, to compare contrasts of interest. Multiplicity adjustment was done according to the Benjamini and Hochberg procedure.

Treatment effects are presented in Table 6 as mean effect size estimates with 95% CIs, test-statistics and multiple comparison adjusted p-values (=FDR values).
Missing values for insulin (n=1), GLP-1 (n=4) and glucagon (n=5) were assumed to be missing at random and imputed using multiple imputation with the R-package “mice”. Inferential statistical analyses were done using R version 3.5.2. Descriptive statistics were done with GraphPad Prism8 version 8.0.1(145). Effects were regarded as statistically significant at an FDR-value below 0.05.
Insulin secretion index was calculated on the basis of plasma glucose and insulin sampled during the mixed-meal test using the following formulas: (insulin_{30 min}−insulin_{fasting})/glucose_{30 min} (Matsuda, M., and DeFronzo, R. A. (1999). Diabetes Care 22, 1462-1470).

Laboratory Analysis

Immediate bed-side glucose measurement was performed with point of care testing (Contour XT®, Ascensia Diabetes Care, Switzerland) and then confirmed for the analyses in the central laboratory of the University Hospital Basel, where also routine blood count and chemistry analyses were done. Plasma levels of insulin, c-peptide, glucagon, and GLP-1 were measured with ELISAs by Mercodia AB, Uppsala, Sweden (assay #10-1113-01, 10-1136-01, 10-1271-01, and 10-1278-01 respectively) according to the manufacturer's instructions.

Purification of Blood Monocytes

Blood samples were taken before and 60 minutes after meal intake. Due to scheduling conflicts, samples from two patients could not be processed. These samples are thus assumed to be missing at random. Of note, samples from patient 3 (sleeve gastrectomy) had already been processed for RNA sequencing before the final change of protocol. Sequencing data obtained from this patient were thus kept for the purpose of these analyses. Peripheral blood mononuclear cells were obtained by Ficoll density gradient centrifugation (Lymphoprep Fresenius Kabi, Norway). For 9 out of 11 patients, an additional step was added after density gradient centrifugation to pre-enrich monocytes by negative selection with CD3 human MicroBeads (#130-050-101; Miltenyi Biotec). The obtained cellular monocyte fractions were then incubated with FC-Block for 15 minutes at room temperature [(Human FcR TruStain FcX (Biolegend) REF 422302, compatible with flow cytometric staining with anti-human CD16 (clone 3G8)], and subsequently labeled with the following antibodies: APC Mouse anti-Human CD3 [HIT3a alpha] (#555342; BD Biosciences), FITC Mouse Anti-Human CD19 [HIB19 RUO] (#560994; BD Biosciences), PE-Cy5 Mouse Anti-Human CD56 [B159 RUO] (#560842; BD Biosciences), PE Mouse Anti-Human CD16 [3G8 RUO] (#560995; BD Biosciences), APC-Cy7 Mouse Anti-Human CD14 [MφP-9 RUO] (#561709; BD Biosciences).

Cells were then further enriched into the three monocyte subsets by fluorescence-activated cell sorting on a BD FACS-Aria III. Classical monocytes were defined as: single, live, CD56− CD19−CD3−CD14+CD16−, Intermediate monocytes were defined as: single, live CD56−CD19− CD3−CD14+CD16+ and non-classical monocytes were defined as: single, live CD56−CD19− CD3−CD14−CD16+. Bulk monocytes were defined as a combination-gate of the three subsets. For a representative example of the gating strategy see FIG. 13.

Cytokine-Secretion Assays

Bulk monocytes obtained as described above were resuspended in fresh medium (RPMI-1640 (containing 11 mM Glucose, #31870-025; GIBCO) containing 200 U/ml penicillin, 200 U/ml streptomycin (Pen/Strep, #15140; GIBCO), 2 mM Glutamax (#35050061; GIBCO), 1 mM NaPyruvat (#11360-039; GIBCO), 1×MeM (#11140050; Sigma) and 10% FCS (#10500; Invitrogen)) at a density of 50'000 cells/well in a standard 96-well U-bottom plate. For each patient-visit sample, monocytes pre- and post-liquid mixed-meal were either left unstimulated (baseline condition) or activated with LPS (LPS-SM, #tlrl-smlps, Invivogen=activated condition) at a concentration of 5 ng/ml in duplicates. Thus, treated cells were then incubated for a period of 18 hours at 37° C. and 5% CO₂ followed by harvesting of cell supernatants. Cytokine concentrations were determined in cell supernatants using the V-PLEX Human Proinflammatory Panel II (4-Plex) kit (Mesoscale Discovery) according to the manufacturer's instructions (Alternate Protocol 1, Extended Incubation). Two patients had to be excluded from analysis due to technical problems.

RNA Extraction

Total RNA was extracted from 500'000 bulk monocytes, isolated as described above using the Nucleo Spin RNA II Kit (Machery Nagel). Integrity of isolated total RNA was assessed on a 2100 Bioanalyzer System (Agilent Technologies, USA) with the Agilent RNA 6000 Pico Kit as recommended by the manufacturer. RNA concentration was determined with the Quant-iT RiboGreen RNA Assay Kit (Thermo Fisher Scientific, USA).

Sample Preparation and RNA Sequencing

Purification of mRNA by poly(A)-selection, mRNA fragmentation and cDNA library preparation for RNA-Seq were performed using the TruSeq Stranded mRNA HT Sample Preparation Kit (Illumina, USA) according to the manufacturer's protocol. Before sequencing, quality and quantity analysis of prepared cDNA libraries were done on a Fragment Analyzer System (Advanced Analytical Technologies, USA). cDNA libraries were sequenced as single-end reads with 125 cycles on a HiSeq2500 sequencing system (Illumina, USA) at the Genomics Facility Basel. Of note, 2 samples failed during library preparation.

RNAseq Computational Analysis

Initial quality checks were performed with FastQC version 0.11.4 (www.bioinformatics.babraham.ac.uk/projects/fastqc/). Adaptor clipping and quality-trimming of sequences was performed using Trimmomatic version 0.36 (Bolger, A. M., Lohse, M., and Usadel, B. (2014). Bioinformatics 30, 2114-2120) and reads were aligned to the GRCh38 reference genome using the splice aware aligner STAR version 2.7.0 (Dobin, A., et al. (2013). Bioinformatics 29, 15-21). Count-tables were produced using HTSeq version 0.6.1 (Anders, S., Pyl, P. T., and Huber, W. (2015). Bioinformatics 31, 166-169). Subsequent analyses were performed in R (v3.5.3, www.r-project.org) using the DESeq2 package version 1.20.0 (Love, M. I., Huber, W., and Anders, S. (2014). Genome Biol 15, 550) and WCGNA package version 1.66 (Langfelder, P., and Horvath, S. (2008). BMC Bioinformatics 9). GO- and REACTOME pathway analysis was performed using the R package clusterProfiler version 3.10.1 (Yu, G., Wang, L. G., Han, Y., and He, Q. Y. (2012). OMICS 16, 284-287). Custom input pathway analysis testing the enrichment of a gene signature defining an “Endotoxin Tolerance phenotype” was analysed using the R package “pathfindR” version 1.3.0 (Ulgen, E., Ozisik, O., and Sezerman, O. U. (2018). bioRxiv, 272450). Unsupervised hierarchical clustering of the 20 top variably expressed genes and visualization thereof was performed using the R package pheatmap version 1.0.12 (Kolde, R. (2019). pheatmap: Pretty Heatmaps. R package version 1.0.12).

Analysis of Mean Trend in Gene Expression

Applying linear mixed effect models using the genes themselves as random effects, the inventors were able to analyze the mean trend of regularized log fold changes within each cluster identified by the pheatmap function's unsupervised hierarchical cluster algorithm (R «pheatmap» package v1.0.12 (Kolde, R. (2019). pheatmap: Pretty Heatmaps. R package version 1.0.12)). To obtain regularized log fold changes, the inventors transformed count values with the r log function included in the previously mentioned R package «DESeq2» (Love, M. I., Huber, W., and Anders, S. (2014). Genome Biol 15, 550). Regularized log fold changes were then obtained by subtraction of the base mean for each gene. Generation of a dummy variable combining meal and hypo status allowed us to estimate beta-coefficients and p-values for the pre-meal-no-hypo, pre-meal-hypo, post-meal-no-hypo and post-meal-hypo state.

TABLE 1
Study schedule

| Visit | Screening | (study day 0) | study day 1 | study day 2 | study day 3 |
|---|---|---|---|---|---|
| Inclusion/exclusion criteria | x | | | | |
| informed consent | x | | | | |
| demographics | x | | | | |
| medical history | x | | | | |
| concomitant medication | x | | x | x | x |
| physical examination | x | | x | x | x |
| vital signs | x | | x | x | x |
| CBC | x | | x | x | x |
| immune cell subpopulation^{#} | | | x | x | x |
| serum chemistry^{$} | x | | x | x | x |
| HbA1c | x | | | | |
| glucose, insulin, c-peptide* | | | x | x | x |
| incretins (GLP1, PYY, Ghrelin) * | | | x | x | x |
| Leptin, Adiponectin^{#} | | | x | x | x |
| IL6, IL-1Ra, CRP * | | | x | x | x |
| adverse events | | | x | x | x |
| trial medication^{§} | | | | | |
| Jardiance ® vs. Placebo | | | x | x | x |
| Kineret ® vs. Placebo | | | x | x | x |
| mixed-meal-test (3 h)^{4,13} | | | x | x | x |
| Freestyle libre ® (glucose flash | | x | | | |
| monitoring system) by choice | | | | | |
| SF-36 Scale | x | | | | |
| Dumping Rating Scale | x | | | | |
| Sigstad Scoring System | | | x | x | x |
| Stanford Sleepiness Scale | | | x | x | x |
| Edinburgh Hypoglycaemia Scale | | | x | x | x |

^{$}Serum chemistry includes: potassium, sodium, creatinine, liver transaminases (ALT, AST), alkaline phosphatase, and y-glutamyl transferase
*repetitive sampling during mixed-meal-test at baseline, 0, 30, 60, 90, 120, 180 minutes. If hypoglycaemia occurs, sampling will be repeated (last sampling), hypoglycaemia will be corrected and the test will be discontinued
^{§}oral trial medication (placebo or empagliflozin) will be administered 2 hours and subcutaneous study medication (placebo or anakinra) 3 hours before start of mixed-meal test
^{#}immune cell subpopulation will be taken at baseline and 60 minutes after ingestion of mixed-meal-test

TABLE 2
Patient Baseline Characteristics. Baseline characteristics of the 12 study
participants. Given are median values and interquartile ranges.

| baseline characteristics | values | |
|---|---|---|
| sex | | |
| female (n, %) | 9 | (75%) |
| male (n, %) | 3 | (25%) |
| age (years) | 43.5 | (35.3-52.5) |
| time since bariatric surgery (months) | 41.6 | (26.2-67) |
| height (cm) | 165 | (160.8-171) |
| preoperative weight (kg) | 118.5 | (105.5-128) |
| current weight (kg) | 75.7 | (69.8-82.2) |
| preoperative body-mass index (kg/m²) | 42.1 | (39.5-45.6) |
| current body-mass index (kg/m²) | 26.9 | (24.2-29.7) |
| systolic blood pressure (mmHg) | 109 | (97-127) |
| diastolic blood pressure (mmHg) | 75 | (65-80) |
| heart rate (/min) | 76 | (64-89) |
| glycated hemoglobin A1c (%) | 5.1 | (4.67-5.2) |
| fasting glucose (mmol/l) | 4.5 | (4.3-4.9) |
| leucocytes (G/I) | 6.72 | (5.25-8.09) |
| C-reactive protein (mg/l) | 0.6 | (0.37-2.3) |

TABLE 3
Details of hypoglycemic events. Given are all symptomatic hypoglycemic events
for each subject, respective study day and treatment allocation. Regular assessment for
symptoms according to Edinburgh Hypoglycemia Scale was done and rated from 0 (=no
symptoms) to 3 (=severe symptoms) for each symptom and categorized as autonomous (a =
sweating, b = palpitations, c = shaking, d = hunger), or neuroglycopenic (e = confusion, f =
drowsiness, g = odd behaviour, h = speech difficulty, i = incoordination).

| subject | day | treatment | symptoms | total |
|---|---|---|---|---|
| 4 | 1 | anakinra | g2, i2, h2 | 6 |
| 5 | 2 | anakinra | a3, d1, e2g2 ,h2 | 10 |
| 4 | 2 | placebo | a2, d2 | 4 |
| 4 | 2 | placebo | e2, h2, g2 | 6 |
| 5 | 1 | placebo | a3, c3, h1, g3 | 10 |
| 6 | 1 | placebo | f2, g2, h1, j1 | 6 |
| 8 | 1 | placebo | e1, f3, g1 | 5 |
| 9 | 2 | placebo | e2, f3, g1 | 6 |
| 10 | 3 | placebo | f3, g1, c2, b2 | 8 |
| 14 | 3 | placebo | a1, f3, g1 ,i1 | 6 |
| 13 | 1 | empagliflozin | a3, e2, g2, h2 | 9 |
| 6 | 2 | empagliflozin | e2, f2, g2, h2 | 8 |

TABLE 4
Edinburgh Hypoglycemia Scale Scores. Given are all Edinburgh hypoglycemia scale scores for each subject,
respective study day and treatment allocation. Regular assessment for symptoms according to Edinburgh
Hypoglycemia Scale was done at timepoint −180, −120, 0, 60, 90, 120, 150 and 180 minutes. Apparent
symptoms were rated from 0 (=no symptoms) to 3 (=severe symptoms) for each symptom and categorized
as autonomous (a = sweating, b = palpitations, c = shaking, d = hunger), or neuroglycopenic (e = confusion,
f = drowsiness, g = odd behaviour, h = speech difficulty, i = incoordination).

| | | | timepoint (min) | | | | | | | |
|---|---|---|---|---|---|---|---|---|---|---|
| treatment | patient | visit | −180 | −120 | 0 | 60 | 90 | 120 | 150 | 180 |
| placebo | 1 | 3 | 0 | 0 | 0 | 0 | 0 | 0 | 0 | 0 |
| empagliflozin | 1 | 2 | 0 | 0 | 0 | 0 | 0 | 0 | 0 | 0 |
| anakinra | 1 | 1 | 0 | 0 | 0 | f1 | 0 | f2 | f3 | f3 |
| placebo | 2 | 2 | 0 | 0 | 0 | f2 | f1 | f1 | 0 | 0 |
| empagliflozin | 2 | 1 | 0 | 0 | 0 | 0 | c1 | c1 | c1 | 0 |
| anakinra | 2 | 3 | 0 | 0 | 0 | f2 | f3 | f3 | f2 | f2 |
| placebo | 3 | 3 | 0 | 0 | f3 | f3 | f3 | f3 | f3 | f1 |
| empagliflozin | 3 | 1 | 0 | 0 | j1 | f3 | 0 | 0 | 0 | 0 |
| anakinra | 3 | 2 | 0 | j1, f2 | f1 | f3, j1, k1 | f1 | 0 | 0 | 0 |
| placebo | 4 | 2 | f1 | f1, d2 | f1 | 0 | e2, g2, h2 | a2, d2 | 0 | 0 |
| empagliflozin | 4 | 3 | f1 | f2 | f2 | f1 | f2 | f1 | 0 | 0 |
| anakinra | 4 | 1 | d2, f1 | c1, d1, f1 | j2 | c2, f2 | g2, i2, h2 | f2 | 0 | 0 |
| placebo | 5 | 1 | d2 | d2, f1 | d3, f1 | f2 | f2 | f1 | 0 | a3, c3, d1, g3 |
| empagliflozin | 5 | 3 | 0 | 0 | f1 | f2, k1 | f3, k1 | f3 | f3 | f1 |
| anakinra | 5 | 2 | 0 | a2 | d2 | f1 | f1 | f1 | a3, d1, e2 | f2 |
| | | | | | | | | | g2, h2 | |
| placebo | 6 | 1 | 0 | f1 | f1 | f2, b1, k1 | f2, g2 ,h1, j1 | c2 | f2, c1 | 0 |
| empagliflozin | 6 | 2 | 0 | f1 | f2 | 0 | e2, c2 | e2, f2, j2, h2 | 0 | f2 |
| anakinra | 6 | 3 | 0 | 0 | 0 | f1 | c2, f1 | f1 | 0 | 0 |
| placebo | 7 | 1 | 0 | 0 | 0 | 0 | 0 | 0 | 0 | 0 |
| empagliflozin | 7 | 2 | 0 | 0 | 0 | 0 | 0 | 0 | 0 | 0 |
| anakinra | 7 | 3 | 0 | 0 | 0 | 0 | 0 | 0 | 0 | 0 |
| placebo | 8 | 1 | f1 | f1 | f1 | f3 | f1 | f3, e1, g1 | f2 | f1 |
| empagliflozin | 8 | 3 | 0 | f3 | f1 | f1 | d2, f2 | d2, f2 | d2, f1 | d2, f1 |
| anakinra | 8 | 2 | f1 | f3 | f1 | f3 | f3 | f3 | f1 | f1 |
| placebo | 9 | 2 | f2 | f3 | f2 | d1, f1 | d1, f1 | f1 | e2, f3, g1 | f1 |
| empagliflozin | 9 | 3 | f2 | d2, f1 | d2, f1 | k1, f1 | f2 | f2, d1, a2 | f1, d1, a1 | f3, j1 |
| anakinra | 9 | 1 | f2 | f2 | 0 | 0 | 0 | 0 | 0 | 0 |
| placebo | 10 | 3 | f1 | f3, j1 | f3 | f2, | f1, j1 | f2 | f1, b2, c2, | f3, |
| | | | | | | | | | | g1, c2, b2 |
| empagliflozin | 10 | 1 | f1 | f1 | f2 | j1, f1 | j1, f1 | 0 | f2, g2, | 0 |
| anakinra | 10 | 2 | 0 | 0 | 0 | f3 | f1, j1 | j2, f1 | f2, j2, c1 | 0 |
| placebo | 12 | 3 | f1 | f2 | f2 | f2 | f1 | f1 | f1 | f3 |
| empagliflozin | 12 | 1 | 0 | 0 | 0 | f3 | 0 | 0 | a2 | 0 |
| anakinra | 12 | 2 | 0 | 0 | f1 | f2 | f2 | 0 | 0 | 0 |
| placebo | 13 | 2 | 0 | 0 | 0 | 1a, 1b, 1f | 0 | 3f, 1h | 2f | if |
| empagliflozin | 13 | 1 | h1 | h1 | 0 | a2 | f2 | a3, e2, | 0 | al |
| | | | | | | | | h2, g2 | | |
| anakinra | 13 | 3 | 0 | 0 | f1 | f1 | 0 | f1 | f1 | 0 |
| placebo | 14 | 3 | 0 | 0 | 0 | 0 | 0 | f2 | f3, g1 i1 | 0 |
| | | | | | | | | | a1 | |
| empagliflozin | 14 | 1 | 0 | 0 | 0 | b1 | f3 | f3, s1, j1 | f3, s1, j1 | f2, j1 |
| anakinra | 14 | 2 | 0 | 0 | 0 | b1 | f1 | a2, f1 | a2, f3 | a2, f2 |

TABLE 5
Adverse events during study period

| | adverse | | | | | |
|---|---|---|---|---|---|---|
| subject | event | intensity | therapy | action | outcome | relation |
| 4 | constipation | mild | drug | discontinuation | recovered | unlikely |
| | | | | of loperamid | without sequel | |
| 5 | constipation | | | | | |
| 5 | herpes labialis | moderate | none | none | recovered | unrelated |
| | | | | | without sequel | |
| 5 | respiratory | moderate | none | none | recovered | unrelated |
| | upper airway | | | | without sequel | |
| | infection | | | | | |
| 6 | gastroenteritis | moderate | drug | none | recovered | unrelated |
| | | | | | without sequel | |
| 6 | abdominal | mild | drug | none | recovered | unrelated |
| | pain | | | | without sequel | |
| 7 | headaches | mild | none | none | recovered | possible |
| | | | | | without sequel | |
| 8 | sore throat | mild | none | none | recovered | unrelated |
| | | | | | without sequel | |
| 8 | symptomatic | moderate | other | none | recovered | unlikely |
| | hypoglycemia | | | | without sequel | |
| 9 | bad dreams | mild | none | none | recovered | unrelated |
| | | | | | without sequel | |
| 9 | light sore | mild | none | none | recovered | unrelated |
| | throat | | | | without sequel | |
| 10 | abdominal | moderate | none | none | recovered | unrelated |
| | pain | | | | without sequel | |
| 10 | diarrhea | moderate | none | none | recovered | possible |
| | | | | | without sequel | |
| 13 | diarrhea | moderate | drug | none | recovered | unrelated |
| | | | | | without sequel | |
| 13 | abdominal | mild | none | none | recovered | unrelated |
| | cramps | | | | without sequel | |
| 14 | bowel pain | mild | none | none | recovered | unrelated |
| | | | | | without sequel | |
| 14 | upper | moderate | none | none | recovered | unlikely |
| | abdominal | | | | without sequel | |
| | pain | | | | | |
| 14 | presyncope | moderate | none | none | recovered | unlikely |
| | after meal | | | | without sequel | |

TABLE 6
Likelihood Ratio Test results, effect size estimates, confidence-intervals and FDR-values of Linear-Mixed Effect Model Calculations.
FIG. 5b

| | | Chi-square | | | | |
|---|---|---|---|---|---|---|
| | | test | | | | |
| outcome variable | fixed effect tested in LRT | statistic | df | pval LRT | | |
| number of events | treatment effect | 8.7183 | 2 | 0.01279 | | * |
| contrasts | contrast designation | estimate | lower CI | upper CI | FDR-value | |
| 1 | empagliflozin vs placebo | −2.882 | −5.455 | −0.31 | 0.0374692 | * |
| 2 | anakinra vs placebo | −2.882 | −5.455 | −0.31 | 0.0374692 | * |

FIG. 5c

| | | Chi-square | | | | |
|---|---|---|---|---|---|---|
| | | test | | | | |
| outcome variable | fixed effect tested in LRT | statistic | df | pval LRT | | |
| glucose | treatment effect | 12.148 | 2 | 0.002302 | | ** |
| contrasts | contrast designation | estimate | lower CI | upper CI | FDR-value | |
| 1 | empagliflozin vs placebo | −0.472 | −0.828 | −0.117 | 0.0142 | * |
| 2 | anakinra vs placebo | 0.133 | −0.224 | 0.49 | 0.4637 | ns |

FIG. 5d

| | | Chi-square | | | | |
|---|---|---|---|---|---|---|
| | | test | | | | |
| outcome variable | fixed effect tested in LRT | statistic | df | pval LRT | | |
| insulin | treatment effect | 37.048 | 2 | 9.02E−09 | | *** |
| contrasts | contrast designation | estimate | lower CI | upper CI | FDR-value | |
| 1 | empagliflozin vs placebo | −0.852 | −1.125 | −0.58 | <0.0001 | **** |
| 2 | anakinra vs placebo | −0.266 | −0.538 | 0.00705 | 0.0562 | ns |

FIG. 5e

| | | Chi-square | | | | |
|---|---|---|---|---|---|---|
| | | test | | | | |
| outcome variable | fixed effect tested in LRT | statistic | df | pval LRT | | |
| insulin AUC | treatment effect | 15.432 | 2 | 0.0004457 | | *** |
| contrasts | contrast designation | estimate | lower CI | upper CI | FDR-value | |
| 1 | empagliflozin vs placebo | −19.32 | −28.2 | −10.48 | 0.0004 | *** |
| 2 | anakinra vs placebo | −13.9 | −22.7 | −5.06 | 0.0052 | * |

FIG. 5f

| | | Chi-square | | | | |
|---|---|---|---|---|---|---|
| | | test | | | | |
| outcome variable | fixed effect tested in LRT | statistic | df | pval LRT | | |
| c-peptide | treatment effect | 36.855 | 2 | 9.93E−09 | | **** |
| contrasts | contrast designation | estimate | lower CI | upper CI | FDR-value | |
| 1 | empagliflozin vs placebo | −0.2237 | −0.294 | −0.1531 | <0.0001 | **** |
| 2 | anakinra vs placebo | −0.0912 | −0.162 | −0.0207 | 0.0114 | * |

FIG. 8b

| | | Chi-square | | | | |
|---|---|---|---|---|---|---|
| | | test | | | | |
| outcome variable | fixed effect tested in LRT | statistic | df | pval LRT | | |
| rlogFC | dummy variable coding for both | 536.41 | 3 | <2.2e−16 | | **** |
| | meal and hypoglycemia effect | | | | | |
| contrasts | contrast designation | estimate | lower CI | upper CI | FDR-value | |
| 1 | Post-meal, no-hypo vs pre- | 0.1146 | 0.0997 | 0.1296, | <0.0001 | **** |
| | meal, no-hypo | | | | | |
| 2 | Post-meal, hypo vs post-meal, | 0.1543 | 0.1295 | 0.1791 | <0.0001 | **** |
| | no-hypo | | | | | |

FIG. 8c

| | | Chi-square | | | | |
|---|---|---|---|---|---|---|
| | | test | | | | |
| outcome variable | fixed effect tested in LRT | statistic | df | pval LRT | | |
| rlogFC | dummy variable coding for both | 684.07 | 3 | <2.2e−16 | | **** |
| | meal and hypoglycemia effect | | | | | |
| contrasts | contrast designation | estimate | lower CI | upper CI | FDR-value | |
| 1 | post-meal, no-hypo vs pre- | −0.1552 | −0.17 | −0.14035 | <0.0001 | **** |
| | meal, no-hypo | | | | | |
| 2 | post-meal, hypo vs post-meal, | −0.0974 | −0.122 | −0.07287 | <0.0001 | **** |
| | no-hypo | | | | | |

FIG. 9a

| | | Chi-square | | | | |
|---|---|---|---|---|---|---|
| | | test | | | | |
| outcome variable | fixed effect tested in LRT | statistic | df | pval LRT | | |
| IL-1 beta | interaction between meal, | 18.383 | 7 | 0.01036 | | * |
| | treatment and hypoglycemia | | | | | |
| | effect | | | | | |
| contrasts | contrast designation | estimate | lower CI | upper CI | FDR-value | |
| 1 | placebo, post-meal, no-hypo vs | 3196.37 | 1692 | 4701 | 0.0011 | ** |
| | placebo, pre-meal, no-hypo | | | | | |
| 2 | anakinra, post-meal, no-hypo vs | −2426.73 | −3762 | −1091.1 | 0.0057 | * |
| | placebo, post-meal, no-hypo | | | | | |
| 3 | empagliflozin, post-meal, no- | −3395.36 | −4720 | −2071.1 | 0.0002 | ** |
| | hypo vs placebo, post-meal, no- | | | | | |
| | hypo | | | | | |
| 4 | placebo, post-meal, hypo vs | −3529.47 | −5008 | −2051.1 | 0.0002 | ** |
| | placebo, post-meal, no-hypo | | | | | |

FIG. 9b

| | | Chi-square | | | | |
|---|---|---|---|---|---|---|
| | | test | | | | |
| outcome variable | fixed effect tested in LRT | statistic | df | pval LRT | | |
| IL-6 | interaction between meal, | 29.002 | 7 | 0.0001446 | | *** |
| | treatment and hypoglycemia | | | | | |
| | effect | | | | | |
| contrasts | contrast designation | estimate | lower CI | upper CI | FDR-value | |
| 1 | placebo, post-meal, no-hypo vs | 297773.8 | 13203 | 26344 | <0.0001 | **** |
| | placebo, pre-meal, no-hypo | | | | | |
| 2 | anakinra, post-meal, no-hypo vs | −17208.1 | −23041 | −11375 | <0.0001 | **** |
| | placebo, post-meal, no-hypo | | | | | |
| 3 | empagliflozin, post-meal, no- | −18937.7 | −24721 | −13155 | <0.0001 | **** |
| | hypo vs placebo, post-meal, no- | | | | | |
| | hypo | | | | | |
| 4 | placebo, post-meal, hypo vs | −18229 | −24685 | −11773 | <0.0001 | **** |
| | placebo, post-meal, no-hypo | | | | | |

FIG. 9c

| | | Chi-square | | | | |
|---|---|---|---|---|---|---|
| | | test | | | | |
| outcome variable | fixed effect tested in LRT | statistic | df | pval LRT | | |
| TNFalpha | interaction between meal, | 22.796 | 7 | 0.00185 | | ** |
| | treatment and hypoglycemia | | | | | |
| | effect | | | | | |
| contrasts | contrast designation | estimate | lower CI | upper CI | FDR-value | |
| 1 | placebo, post-meal, no-hypo vs | 491.883 | 293 | 691 | <0.0001 | **** |
| | placebo, pre-meal, no-hypo | | | | | |
| 2 | anakinra, post-meal, no-hypo vs | −452.659 | −629 | −276 | <0.0001 | **** |
| | placebo, post-meal, no-hypo | | | | | |
| 3 | empagliflozin, post-meal, no-hypo | −453.496 | −629 | −278 | <0.0001 | **** |
| | vs placebo, post-meal, no-hypo | | | | | |
| 4 | Placebo, Post-meal, hypo vs | −465.4 | −661 | −270 | 0.0002 | *** |
| | Placebo, Post-meal, no-hypo | | | | | |

FIG. 9d

| | | Chi-square | | | | |
|---|---|---|---|---|---|---|
| | | test | | | | |
| outcome variable | fixed effect tested in LRT | statistic | df | pval LRT | | |
| IL-1beta | interaction between activation | 4.7913 | 1 | 0.0286 | | * |
| | status and hypoglycemia effect | | | | | |
| contrasts | contrast designation | estimate | lower CI | upper CI | FDR-value | |
| 1 | LPS, no-hypo vs ns, no-hypo | 2541 | 1525 | 3556 | <0.0001 | **** |
| 2 | LPS, hypo vs LPS, no-hypo | −2416 | −4063 | −769 | 0.0089 | ** |

FIG. 9e

| | | Chi-square | | | |
|---|---|---|---|---|---|
| | | test | | | |
| outcome variable | fixed effect tested in LRT | statistic | df | pval LRT | |
| IL-6 | interaction between activation | 3.0191 | 1 | 0.082 | ns |
| | status and hypoglycemia effect | | | | |

FIG. 9f

| | | Chi-square | | | | |
|---|---|---|---|---|---|---|
| | | test | | | | |
| outcome variable | fixed effect tested in LRT | statistic | df | pval LRT | | |
| TNFalpha | interaction between activation | 5.507 | 1 | 0.01894 | | * |
| | status and hypoglycemia effect | | | | | |
| contrasts | contrast designation | estimate | lower CI | upper CI | FDR-value | |
| 1 | LPS, no-hypo vs ns, no-hypo | 522.3 | 316 | 728.1 | <0.0001 | **** |
| 2 | LPS, hypo vs LPS, no-hypo | −404.5 | −730 | −78.9 | 0.0308 | * |

FIG. 11a

| | | Chi-square | | | | |
|---|---|---|---|---|---|---|
| | | test | | | | |
| outcome variable | fixed effect tested in LRT | statistic | df | pval LRT | | |
| number of events ≥ | treatment effect | 6.0584 | 2 | 0.04835 | | * |
| Edinburgh composite | | | | | | |
| core of 6 | | | | | | |
| contrasts | contrast designation | estimate | lower CI | upper CI | FDR-value | |
| 1 | empagliflozin vs placebo | −2.429 | −4.946 | 0.088 | 0.0780 | ns |
| 2 | anakinra vs placebo | −2.429 | −4.946 | 0.088 | 0.0780 | ns |

FIG. 11b

| | | Chi-square | | | |
|---|---|---|---|---|---|
| | | test | | | |
| outcome variable | fixed effect tested in LRT | statistic | df | pval LRT | |
| Sigstadt score | treatment effect | 1.6916 | 2 | 0.4292 | ns |

FIG. 11c

| | | Chi-square | | | |
|---|---|---|---|---|---|
| | | test | | | |
| outcome variable | fixed effect tested in LRT | statistic | df | pval LRT | |
| Stanfort sleepiness scale | treatment effect | 0.9028 | 2 | 0.6368 | ns |
| score | | | | | |

FIG. S5a

| | | Chi-square | | | |
|---|---|---|---|---|---|
| | | test | | | |
| outcome variable | fixed effect tested in LRT | statistic | df | pval LRT | |
| GLP-1 | hypoglycemia effect | 1.5831 | 1 | 0.2083 | ns |

FIG. 12b

| | | Chi-square | | | |
|---|---|---|---|---|---|
| | | test | | | |
| outcome variable | fixed effect tested in LRT | statistic | df | pval LRT | |
| glucagon | hypoglycemia effect | 3.5906 | 1 | 0.05811 | ns |

FIG. S5b [outlier removed]

| | | Chi-square | | | |
|---|---|---|---|---|---|
| | | test | | | |
| outcome variable | fixed effect tested in LRT | statistic | df | pval LRT | |
| glucagon | hypoglycemia effect | 0 | 1 | 0.9987 | ns |

FIG. 12c

| | | Chi-square | | | | |
|---|---|---|---|---|---|---|
| | | test | | | | |
| outcome variable | fixed effect tested in LRT | statistic | df | pval LRT | | |
| GLP-1 AUC | hypoglycemia effect | 4.3962 | 1 | 0.03602 | | * |
| contrasts | contrast designation | estimate | lower CI | upper CI | FDR-value | |
| 1 | hypo vs no-hypohypo-no hypo | 2.221 | 0.114 | 2.8 | 0.0345 | * |

FIG. 12c [outlier removed]

| | | Chi-square | | | |
|---|---|---|---|---|---|
| | | test | | | |
| outcome variable | fixed effect tested in LRT | statistic | df | pval LRT | |
| GLP-1 AUC | hypoglycemia effect | 3.6905 | 1 | 0.05472 | ns |

FIG. 12d

| | | Chi-square | | | | |
|---|---|---|---|---|---|---|
| | | test | | | | |
| outcome variable | fixed effect tested in LRT | statistic | df | pval LRT | | |
| glucagon AUC | hypoglycemia effect | 5.7789 | 1 | 0.01622 | | * |
| contrasts | contrast designation | estimate | lower CI | upper CI | FDR-value | |
| 1 | hypo vs no-hypo | 2.85 | 0.73 | 4.97 | 0.0103 | * |

FIG. 12d [outlier removed]

| | | Chi-square | | | |
|---|---|---|---|---|---|
| | | test | | | |
| outcome variable | fixed effect tested in LRT | statistic | df | pval LRT | |
| glucagon AUC | hypoglycemia effect | 1.1875 | 1 | 0.2762 | ns |

FIG. 12e

| | | Chi-square | | | |
|---|---|---|---|---|---|
| | | test | | | |
| outcome variable | fixed effect tested in LRT | statistic | df | pval LRT | |
| GLP-1 | treatment effect | 0.8005 | 2 | 0.6702 | ns |

FIG. S5f

| | | Chi-square | | | |
|---|---|---|---|---|---|
| | | test | | | |
| outcome variable | fixed effect tested in LRT | statistic | df | pval LRT | |
| glucagon | treatment effect | 0.32493 | 2 | 0.197 | ns |

FIG. 12g

| | | Chi-square | | | |
|---|---|---|---|---|---|
| | | test | | | |
| outcome variable | fixed effect tested in LRT | statistic | df | pval LRT | |
| GLP-1 AUC | treatment effect | 2.639 | 2 | 0.2673 | ns |

FIG. 12h

| | | Chi-square | | | |
|---|---|---|---|---|---|
| | | test | | | |
| outcome variable | fixed effect tested in LRT | statistic | df | pval LRT | |
| glucagon AUC | treatment effect | 1.9111 | 2 | 0.3846 | ns |

Table abbreviations: ns = non-stimulated, LPS = Lipopolysaccharide, GLP-1 = Glucagon-like-peptide 1, hypo = hypoglycemia, pvalLRT = p-value generated by the Likelihood-Ratio Test comparing a full model against a reduced one lacking the comparator of interest.

1. A method for treatment or inhibiting the development of hypoglycaemia after bariatric surgery comprising:
administering a SGLT-2 inhibitor to a subject in need thereof, thereby treating or inhibiting the development of hypoglycaemia after bariatric surgery.

2. The method of claim 1, wherein said bariatric surgery is selected from Roux-Y-gastric bypass, vertical banded gastroplasty surgery, adjustable gastric band, and partial ileal bypass surgery, particularly wherein said bariatric surgery is Roux-Y-gastric bypass.

3. The method of claim 1, wherein said SGLT-2 inhibitor is selected from empagliflozin, canagliflozin, dapagliflozin, ertugliflozin, ipragliflozin, luseogliflozin, sotagliflozin, and tofogliflozin, particularly wherein said SGLT-2 inhibitor is empagliflozin.

4. The method of claim 1, wherein said SGLT-2 inhibitor is administered once a day, particularly three to one hours before a meal, more particularly two hours before a meal.

5. The method of claim 1, wherein said SGLT-2 inhibitor is administered at a dose of 5-20 mg/day.

6. The method of claim 1, wherein the hypoglycaemia is symptomatic hypoglycaemia.

7. A method for treatment or inhibiting the development of hypoglycaemia after bariatric surgery comprising:
administering to a subject in need thereof an interleukin-1-receptor antagonist,
thereby treating or inhibiting the development of hypoglycaemia after bariatric surgery.

8. The method of claim 7, wherein said bariatric surgery is selected from Roux-Y-gastric bypass, vertical banded gastroplasty surgery, adjustable gastric band, and partial ileal bypass surgery, particularly wherein said bariatric surgery is Roux-Y-gastric bypass.

9. The method of claim 7, wherein said interleukin-1-receptor antagonist is anakinra.

10. The method of claim 7, wherein said interleukin-1-receptor antagonist is administered once a day, particularly four to two hours before a meal, more particularly three hours before a meal.

11. The method of claim 7, wherein said interleukin-1-receptor antagonist is administered at a dose of 50-200 mg/day.

12. The method of claim 7, wherein the hypoglycaemia is symptomatic hypoglycaemia.

13. A method for treatment or inhibiting the development of hypoglycaemia, particularly symptomatic hypoglycaemia, after bariatric surgery comprising:
administering to a subject a non-agonist antibody or antibody-like molecule specifically binding to one of
IL-1β or
IL-1 receptor type I
thereby treating or inhibiting the development of hypoglycaemia, particularly symptomatic hypoglycaemia, after bariatric surgery.

14. A method for treatment or inhibiting the development of hypoglycaemia, particularly symptomatic hypoglycaemia, after bariatric surgery comprising:
administering to a subject a NLRP3 inhibitor, thereby treating or inhibiting the development of hypoglycaemia, particularly symptomatic hypoglycaemia, after bariatric surgery.
